(19) United States
(12) Patent Application Publication
(10) Pub. No.: US 2017/0049793 A1
(43) Pub. Date: Feb. 23, 2017

(54) MOLECULAR ACTIVATORS OF THE WNT/BETA-CATENIN PATHWAY

(71) Applicants: Randall T. Moon, Kenmore, WA (US); Travis L. Biechele, Seattle, WA (US); Nathan D. Camp, Seattle, WA (US); Stephen Haggarty, Dorchester, MA (US); Daniel Fass, Winchester, MA (US)

(72) Inventors: Randall T. Moon, Kenmore, WA (US); Travis L. Biechele, Seattle, WA (US); Nathan D. Camp, Seattle, WA (US); Stephen Haggarty, Dorchester, MA (US); Daniel Fass, Winchester, MA (US)

(73) Assignees: THE GENERAL HOSPITAL CORPORATION, Boston, MA (US); UNIVERSITY OF WASHINGTON THROUGH ITS CENTER FOR COMMERCIALIZATION, Seattle, WA (US); MASSACHUSETTS INSTITUTE OF TECHNOLOGY, Cambridge, MA (US)

(21) Appl. No.: 14/701,936

(22) Filed: May 1, 2015

Related U.S. Application Data
(63) Continuation of application No. 13/939,679, filed on Jul. 11, 2013, now abandoned.
Application No. 13/939,679 is a continuation of application No. 13/141,838, filed on Nov. 4, 2011, now abandoned.

(51) Int. Cl. A61K 31/708; A61K 31/353; A61K 31/7048; A61K 31/357; A61K 31/506; A61K 31/4406; A61K 31/167; A61K 31/196; A61K 31/24; A61K 31/4409; A61K 31/472; A61K 31/5377; A61K 31/69; A61K 31/15; A61K 31/495; A61K 31/426; A61K 31/52; A61K 31/404; A61K 31/405; A61K 31/4178; A61K 31/4439; A61K 31/047; A61K 31/695; A61K 31/407; A61K 31/403; A61K 31/421; A61K 31/5383; A61K 31/351; A61K 31/4152; A61K 31/166; A61K 31/585; A61K 31/57; A61K 31/565; A61K 31/365; A61K 31/201; A61K 31/336; A61K 31/4402; A61K 31/015; A61K 31/366; A61K 31/553; A61K 31/5575; A61K 31/55; A61K 31/27; A61K 31/4245; A61K 31/352

(52) U.S. Cl. CPC A61K 31/708; A61K 31/352; A61K 31/353; A61K 31/7048; A61K 31/357; A61K 31/506; A61K 31/4406; A61K 31/167; A61K 31/196; A61K 31/24; A61K 31/4409; A61K 31/472; A61K 31/5377; A61K 31/69; A61K 31/15; A61K 31/495; A61K 31/426; A61K 31/52; A61K 31/404; A61K 31/405; A61K 31/4178; A61K 31/4439; A61K 31/047; A61K 31/695; A61K 31/407; A61K 31/403; A61K 31/421; A61K 31/5383; A61K 31/351; A61K 31/4152; A61K 31/166; A61K 31/585; A61K 31/57; A61K 31/565; A61K 31/365; A61K 31/201; A61K 31/336; A61K 31/4402; A61K 31/015; A61K 31/366; A61K 31/553; A61K 31/5575; A61K 31/55; A61K 31/27; A61K 31/4245

(57) ABSTRACT

The present invention is directed toward a method of treating a subject for a condition mediated by aberrant Wnt/β-catenin signaling by selecting a subject with a condition mediated by aberrant Wnt/β-catenin signaling and administering to the selected subject a compound selected from the group consisting of those set forth in Table 1, Table 2, and a pharmaceutically acceptable salt thereof. A method of similarly modulating the Wnt/β-catenin pathway in a subject is also discussed.

### CROSS-REFERENCE TO RELATED APPLICATIONS

This application is a continuation of U.S. patent application Ser. No. 13/939,679 filed Jul. 11, 2013, which is a continuation application of U.S. patent application Ser. No. 13/141,838 filed Nov. 4, 2011, which is a 35 U.S.C. §371 National Phase Entry Application of International Application No. PCT/US2009/069003 filed Dec. 21, 2009, which designates the U.S., and which claims benefit under 35 U.S.C. §119(e) of U.S. Provisional Patent Application Ser. No. 61/140,655, filed Dec. 24, 2008, the contents of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Dec. 6, 2013, is named 030258-001463-C_SL.txt and is 1,435 bytes in size.

### FIELD OF THE INVENTION

This invention relates to molecular activators of the Wnt/β-catenin pathway.

### BACKGROUND OF THE INVENTION

Wnt/β-catenin signaling regulates cell fate and proliferation during development, homeostasis, and disease. The canonical Wnt pathway describes a series of events that occur when Wnt proteins bind to cell-surface receptors of the Frizzled family, causing the receptors to activate Dishevelled family proteins and ultimately resulting in a change in the amount of β-catenin that reaches the nucleus. Dishevelled (DSH) is a key component of a membrane-associated Wnt receptor complex which, when activated by Wnt binding Frizzled, inhibits a second complex of proteins that includes axin, GSK-3, and the protein APC. The axin/GSK-3/APC complex normally promotes the proteolytic degradation of the β-catenin intracellular signaling molecule. After this “β-catenin destruction complex” is inhibited, a pool of cytoplasmic β-catenin stabilizes, and some β-catenin is able to enter the nucleus and interact with TCF/LEF family transcription factors to promote specific gene expression.

Numerous diseases have been linked to aberrant Wnt/β-catenin signaling and several conditions (Moon R T, “WNT and Beta-catenin Signaling: Diseases and Therapies,” Nat Rev Gen 5(9):691-701 (2004)). It is also clear that activation of Wnt/β-catenin signaling may be therapeutic for a variety of other indications including those involving a deficit in stem/progenitor cells. Lithium chloride is currently the only FDA approved small molecule modulator of Wnt/β-catenin signaling. The narrow therapeutic range of lithium combined with the vast number of diseases linked to Wnt/β-catenin signaling begs the discovery of additional small molecule modulators.

The present invention is directed at identifying small molecule modulators of Wnt/β-catenin signaling.

### SUMMARY OF THE INVENTION

One aspect of the present invention is directed toward a method of treating a subject for a condition mediated by aberrant Wnt/β-catenin signaling by selecting a subject with a condition mediated by aberrant Wnt/β-catenin signaling and administering to the selected subject a compound selected from the group consisting of those set forth in Table 1, Table 2, and a pharmaceutically acceptable salt thereof.

Another aspect of the present invention is directed toward a method of activating the Wnt/β-catenin pathway in a subject including selecting a subject in need of Wnt/β-catenin pathway activating and administering to the selected subject a compound selected from the group consisting of those set forth in Table 1, Table 2, and a pharmaceutically acceptable salt thereof.

### BRIEF DESCRIPTION OF THE DRAWINGS

FIGS. 1A-1G illustrate that nuclear β-catenin predicts improved survival in melanoma patients and correlates with decreased tumor proliferation. FIG. 1A is a graph showing that patients with the highest levels of nuclear β-catenin (upper tertile) exhibit an increased survival probability by Kaplan-Meier analysis compared to patients in the middle and lower tertile. This trend was statistically significant by log-rank test. FIG. 1B is a graph showing metastases separated into those with the highest nuclear β-catenin levels (upper 20%, n=46) and those with lower nuclear β-catenin levels (remaining 80%, n=179). Kaplan-Meier analysis showed a significantly increased survival probability in patients with the highest amount of nuclear β-catenin (Gehan-Breslow-Wilcoxon test). FIG. 1C is a graph showing the subset of patients with available data on tumor depth (Breslow thickness) analyzed by Kaplan-Meier survival curves. Tumors were grouped based on the AJCC tumor staging guidelines for tumor depth into T1 (0-1.00 mm, n=35), T2 (1.01-2.00 mm, n=26), T3 (2.01-4.00 mm, n=32) or T4 (>4.00 mm, n=20). The survival curves exhibited an extremely significant trend by log-rank test. FIG. 1D and FIG. 1E are graphs showing tumors grouped by tumor staging depth evaluated for proliferation (FIG. 1D) and for expression of nuclear β-catenin (FIG. 1E). Bars show the mean and standard deviation for each group, while gray dots represent individual tumors. The horizontal dotted lines represent the mean Ki-67 and nuclear β-catenin seen for all tumors in the array. As expected, increasing tumor depth is associated with increased proliferation. By contrast, levels of nuclear β-catenin decrease with increasing tumor depth, suggesting that activation of Wnt/β-catenin signaling is lost with melanoma progression. The trend for both % Ki-67 and nuclear β-catenin was extremely significant by ANOVA (*p<0.002). FIG. 1F is a histogram showing primary tumors stratified into tertiles based on levels of nuclear β-catenin (see FIG. 5), and the distribution of proliferation as measured by % Ki-67 was assessed in each tertile. Patients with the highest levels of nuclear β-catenin (upper tertile, n=39) showed a lower mean % Ki-67 than patients in the middle tertile (n=39) or the lower tertile (n=40). This trend was extremely significant by ANOVA (*p<0.0001). The histogram illustrates that tumors with the lowest levels of nuclear β-catenin (lower tertile) show a clear shift towards higher proliferation compared to patients with the highest levels of nuclear β-catenin (upper tertile). FIG. 1G is a graph showing normalized levels of nuclear β-catenin in primary tumors plotted against proliferation as measured by % Ki-67, and a Deming regression analysis (diagonal line) reveals an extremely significant inverse correlation between levels of nuclear β-catenin and proliferation as measured by Ki-67 (slope=−1.089+/−0.24).

FIGS. 2A-2G illustrate activation of Wnt/β-catenin signaling changes melanoma cell fate. FIG. 2A is a photograph showing B 16 cells expressing GFP, WNT3A or WNT5A isolated at equivalent confluency, spun down and photographed in a 96-well plate, demonstrating the marked difference in pigmentation seen in melanoma cells expressing WNT3A. FIG. 2B shows expression of WNT5A was confirmed by immunoblotting of cell lysates. FIG. 2C shows immunofluorescent staining demonstrating increased nuclear β-catenin in B16 cells expressing WNT3A, consistent with activation of the Wnt/β-catenin pathway. FIG. 2D is a histogram showing conditioned media from B16:GFP, B16:WNT3A and B16:WNT5A cells incubated with a human melanoma cell line stably transduced to express firefly luciferase under the control of a TCF-based Wnt/β-catenin-responsive promoter. Media from B16:WNT3A cells activate the reporter, indicating that these cells secrete active WNT3A. FIG. 2E is a histogram showing expression of the Wnt/β-catenin target gene Axin2 measured by quantitative real-time PCR and normalized to Gapdh. Upregulation of Axin2 is seen in WNT3A cells, indicating activation of the Wnt/β-catenin pathway. FIG. 2F is a histogram showing proliferation of cells expressing GFP, WNT3A or WNT5A was measured by hematocytometer after six days of culture (shaded bars, left y-axis) or by MTT assay after three days of culture (unshaded bars, right y-axis). Bars represent the average and standard deviation of three to six biological replicates. The inhibition of proliferation seen with WNT3A cells is extremely significant by ANOVA with both proliferation assays (*p<0.001). FIG. 2G is a histogram showing cell cycle analysis where cells expressing WNT3A demonstrated a decreased population in phase and an increased population in G1 compared to cells expressing GFP or WNT5A. Bars indicate the average and standard deviation of three biologic replicates, and the data shown are representative of five individual experiments, each with at least three biologic replicates per condition. The changes observed in % G1 and % S with the WNT3A cells is extremely significant by ANOVA (*p<0.001).

FIGS. 3A-3E illustrate that elevation of melanocyte differentiation markers by WNT3A corresponds with decreased tumor growth and metastasis in vivo. FIG. 3A is a heatmap of whole genome expression profiles of WNT3A or WNT5A cell lines compared to gene expression in GFP cells, which served as the reference sample. Three biologic replicates were analyzed for each cell line. The heatmap illustrates the differences between the most significant regulated genes in WNT3A cells compared to WNT5A cells by unpaired t-test. Genes that were among the most significantly regulated in WNT3A cells are listed with normalized fold-change (log 2) compared to GFP cells shown in parentheses. The most significantly regulated genes include known Wnt/β-catenin targets, genes involved in melanocyte and neural crest differentiation, and genes implicated in melanoma prognosis or therapeutics. FIG. 3B is a histogram showing several genes selected for validation using real-time quantitative PCR (qPCR), including genes implicated in melanocyte differentiation (Met, Kit, Sox9, Mitf Si/Gp100), melanoma biology (Trpm1, Kit, Mine, Mlze), and genes that are known Writ target genes (Axing, Met, Sox9). Genes that were upregulated in WNT3A cells by transcriptional profiling are all upregulated by qPCR, while genes that are downregulated in WNT3A cells on the array (Mlze, Mme) are also downregulated by qPCR. Genes upregulated in WNT3A cells are universally downregulated in the WNT5A cells, providing evidence that WNT5A can antagonize transcription of Wnt/β-catenin gene targets in melanoma cells, even in the absence of WNT3A. Data are expressed as log 2-transformed fold-change compared to B16:GFP cells, and are representative of three or more experiments with similar results. FIG. 3C is a histogram showing gene changes induced by WNT3A inhibited upon treatment with β-catenin siRNA (20 nM) compared to control siRNA (20 nM). Data are expressed as log 2-transformed fold-change in cells treated with β-catenin siRNA compared to control siRNA. FIG. 3D is a graph showing tumor explants demonstrating that B16 cells expressing WNT3A form smaller tumors than cells expressing GFP or WNT5A. Data are expressed as the mean and standard deviation from four mice for each tested cell line. The experiment shown is representative of four independent experiments with the same result, all involving at least four mice for each cell line tested. The decrease in tumor size with WNT3A was highly significant by ANOVA at 14 days post-implantation (*p=0.004). FIG. 3E is a plot showing metastases to the popliteal sentinel lymph node bed evaluated by Firefly luciferase assay, demonstrating significantly decreased metastases in tumors expressing WNT3A.

FIGS. 4 A-4D illustrate figures related to tumor microarray analysis. FIG. 4A is a histogram depicting the distribution of nuclear β-catenin staining in the cohort of primary tumors. The bar below shows the cut-offs for the three tertiles used for analysis of survival in FIGS. 1A-1G. FIG. 4B is a histogram depicting survival analysis in metastases. The upper 20% was selected based on both the population distribution and the absolute levels of nuclear-catenin, which correspond roughly with the upper tertile of the population. FIG. 4C is a plot showing levels of nuclear β-catenin compared in primary tumors and metastases/recurrences, showing a decrease in nuclear β-catenin in metastases/recurrences that approximated statistical significance using an unpaired two-tailed t-test. This data supports the hypothesis that Wnt/β-catenin signaling is lost with melanoma progression. FIG. 4D is a plot comparing % Ki-67 with another marker of proliferation, % PCNA. Deming regression analysis gave an extremely significant correlation, with a slope of 1.04 suggesting that proliferation was robustly measured by % Ki-67.

FIGS. 5A-5D illustrate Wnt expression in the context of human melanoma. FIG. 5A is a table showing data from the NCBI Gene Expression Omnibus used to evaluate the expression of Wnt isoforms in benign nevi and melanoma tumors (see also Barrett et al., Nucleic Acids Res. D760-5 (2007), which is hereby incorporated by reference in its entirety). The datasets used include GDS1375 (Talantov et al., Clin. Cancer Res. 11(20):7234-42 (2005), which is hereby incorporated by reference in its entirety) and GDS1989 (Smith et al., Cancer Biol. Ther. 4(9):1018-29 (2005), which is hereby incorporated by reference in its entirety). The primary expression data is shown, and the above table summarizes the data from these two datasets. The data summarization is based on the reported ‘detection call’ of the Affymetrix data used for all three datasets, and the scale indicates the percentage of samples with ‘present’ calls on the expression of the different Wnt isoforms. In the primary data presented above, ‘absent’ calls are faded out. Scoring was as follows: 0 calls were ‘absent’ in all samples; + represents up to 25% of specimens have expression; ++ represents 25-50% of specimens have expression; +++ represents 50-75% of specimens have expression; ++++ represents 75-100% of specimens have expression. Few Wnt isoforms are expressed by melanoma tumors based on this transcriptional profiling, and only wnt3, wnt4, wnt5a and wnt6 were detected in melanomas from both gene datasets. FIG. 5B and FIG. 5C are histograms showing the human melanoma cell lines Mel375 (FIG. 5B) and UACC 1273 (FIG. 5C) were transduced with lentiviral constructs for encoding either GFP or WNT3A. Cells were counted after 3-7 days by hematocytometer and the panels above are representative of multiple experiments with similar results. The bars represent the average and standard deviation from three biologic replicates. P-values for two-tailed t-tests were statistically significant (*p<0.05). Expression of WNT3A also led to a consistent and reproducible decrease in proliferation by MTT assay. No consistent effect on proliferation was seen with expression of WNT5A, again similar to the B 16 cell lines. FIG. 5D is a histogram showing human melanoma cell lines cultured for 3-7 days in the presence of either 10 mM sodium chloride or 10 mM lithium chloride. Proliferation was measured by hematocytometer or MTT assay, and normalized to growth observed in the samples cultured in 10 mM sodium chloride. Lithium chloride inhibited proliferation in all human melanoma cell lines tested.

FIGS. 6A-6F illustrate inhibitors of GSK3 activate Wnt/β-catenin signaling and inhibit proliferation of B16 melanoma cells. FIG. 6A and FIG. 6B are photographs showing immunofluorescent staining of β-catenin demonstrates increased nuclear β-catenin in B16 cells treated with 10 mM lithium chloride or 1 μM BIO compared to control cells treated with 10 mM sodium chloride or DMSO, respectively, consistent with activation of the Wnt/β-catenin pathway by lithium and BIO. FIG. 6C and FIG. 6D are histograms showing quantitative PCR demonstrates increased Axing levels in B 16 cells treated with 10 mM lithium chloride or 1 μM BIO compared to control cells, also consistent with activation of the Wnt/β-catenin pathway by both drugs. FIG. 6E and FIG. 6F are histograms showing representative MTT proliferation assays and demonstrate the decreased proliferation seen in B16 cells treated with 10 mM lithium chloride or 1 μM BIO compared to control cells. Bars represent the mean and standard deviation of three to six biologic replicates. The difference is extremely significant by unpaired two-tailed t-test (p<0.001).

FIGS. 7A-7C illustrate microarray analysis of B16 cells expressing WNT3A and WNTSA. FIG. 7A and FIG. 7B are Venn diagrams which compare the genes upregulated and downregulated in B16 cells expressing WNT3A or WNT5A compared to control 1316 cells expressing GFP, which served as the reference for Agilent whole mouse genome two-channel arrays. Very few genes were regulated by WNTSA compared to WNT3A, consistent with previous results in human melanoma cells. FIG. 7C shows B16 melanoma cells transfected for 72 hours with either control siRNA or siRNA targeting murine β-catenin were analyzed by immunoblotting to assess knockdown of β-catenin protein. The siRNA sequences (SEQ ID NOs: 1-3) tested are on the right. It was found that siRNA #2 and #3 produced marked knockdown of β-catenin protein and for the validation of microarray target genes presented in FIG. 3. Cells were transfected with a pool consisting of 10 nM of siRNA #2 and #3 to minimize off-target effects of each individual siRNA.

FIG. 8 illustrates a model for differentiation therapy using Wnt/β-catenin activators in melanoma. This is a schematic diagram depicting a model of melanoma arising through transformation of differentiated melanocytes and nevus (mole) cells or from melanocytic progenitor cells, taking into account that clinical melanomas arise both from established melanocytic lesions and also de novo (Barnhill et al., Pathology of Melanocytic Nevi and Malignant Melanoma (2004), which is hereby incorporated by reference in its entirety). Based readouts of differentiation such as gene expression profiles, previous studies have found that melanoma progression appears to correlate with the loss of expression of melanocytic markers. Additionally, this model also incorporates the concept of cancer stem cells (or tumor initiating cells) in melanoma (Hendrix et al., Nat Rev. Cancer 7:246 (2007), which is hereby incorporated by reference in its entirety), which give rise to highly proliferative bulk tumor cells, and are themselves highly resistant to conventional chemotherapy in the context of melanoma and other cancer stem cell models. Based on the finding that WNT3A is one of only three factors needed to generate functional melanocytes from embryonic stem cells (Fang et al., Stem Cells 24:1668 (2006), which is hereby incorporated by reference in its entirety), as well as the well-described requirement for Wnt/β-catenin signaling in melanocyte development from animal models (Dorsky et al., Nature 396:370 (1998), which is hereby incorporated by reference in its entirety), the leveraging of this pathway to force cell fate changes in melanoma offers an attractive choice for therapeutic manipulation. The findings herein, as well as other supporting published results (Bachmann et al., Clin. Cancer Res. 11:8606 (2005); Kageshita et al., Br. J. Dermatol. 145:210 (2001), which are hereby incorporated by reference in their entirety) documenting the loss of β-catenin with melanoma progression and decreased survival are depicted below the model. The present data suggests that using activators of Wnt/β-catenin signaling in melanoma can force differentiation in both bulk tumor cells and cancer stem cells to promote cell fates associated with less aggressive tumors through the reactivation of melanocyte-associated transcriptional programs that are downregulated or lost during normal melanoma progression. The goal of differentiation therapy using Wnt/β-catenin activators would be to elicit changes in tumor cell properties through reprogramming of cell, generating tumors that are less aggressive, less proliferative, or potentially more susceptible to currently available melanoma therapies. The availability of several previously FDA-approved activators of Wnt/β-catenin signaling, including riluzole, can facilitate the rapid testing of this therapeutic approach in clinical trials.

### DETAILED DESCRIPTION OF THE INVENTION

One aspect of the present invention is directed toward a method of treating a subject for a condition mediated by aberrant Wnt/β-catenin signaling by selecting a subject with a condition mediated by aberrant Wnt/β-catenin signaling and administering to the selected subject a compound selected from the group consisting of those set forth in Table 1, Table 2, and a pharmaceutically acceptable salt thereof.

In a preferred embodiment of this and other aspects described herein, the subject is human.

The condition which can be treated in accordance with this aspect of the present invention can be any one of the following: cancer (malignant melanoma, colorectal cancer, renal, liver, lung, breast, prostate, ovarian, parathyroid, leukemias, etc), bone mass diseases, fracture repair, FEVR, diabetes mellitus, cord blood transplants, psychiatric disease (e.g., bipolar depression), neurodegenerative disease (Alzheimer's, ALS), hair loss, diseases linked to loss of stem/progenitor cells, conditions improved by increasing stem/progenitor cell populations, HIV, and tooth agenesis.

Another aspect of the present invention is directed toward a method of activating the Wnt/β-catenin pathway in a subject including selecting a subject in need of a Wnt/β-catenin pathway activating and administering to the selected subject a compound selected from the group consisting of those set forth in Table 1, Table 2, and a pharmaceutically acceptable salt thereof.

TABLE 1
Activators/Enhancers

| | |
|---|---|
| Family I | 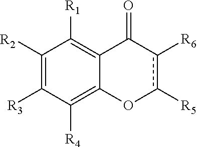 |
| | wherein: |
| | 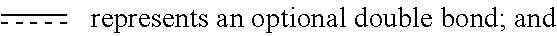 |
| | R₁—R₆ are independently H, halogen, —OH, —OR₇, —C(O)R₇, —OC(O)R₇, |
| | —OC(O)NHR₇, —NH₂, —NHR₇, —NR₇R₈, C₁—C₆ alkyl, C₂—C₆ alkenyl, C₂—C₆ |
| | alkynyl, C₃-C₆ cycloalkyl, monocyclic aryl, arylalkyl, or a mono or bicyclic |
| | heterocycle or heteroaryl containing 1 to 5 heteroatoms selected from the group |
| | consisting of oxygen, nitrogen, and sulfur, each of R₁—R₆ optionally substituted |
| | from 1 to 5 times with substitucnts selected from the group consisting of H, |
| | halogen, —OH, —OR₉, —NH₂, —NHR₉, —NR₉R₁₀, C₁—C₆ alkyl, C₂—C₆ alkenyl, C₂—C₆ |
| | alkynyl, C₃—C₆ cycloalkyl, monocyclic aryl, and arylalkyl, wherein R₇—R₁₀ are |
| | defined as below; |
| | R₇—R₁₀ are independently H, C₁—C₆ alkyl, C₂—C₆ alkenyl, C₂—C₆ alkynyl, C₃—C₆ |
| | cycloalkyl, monocyclic aryl, arylalkyl, or a 3- to 6- membered heterocycle |
| | containing 1 to 5 heteroatoms selected from the group consisting of oxygen, |
| | nitrogen, and sulfur, each of R₇ and R₈ optionally substituted from 1 to 5 times |
| | with substituents selected from the group consisting of H, halogen, —OH, —NH₂, |
| | R₁₁OC(O)—, C₁—C₆ alkyl, C₂—C₆ alkenyl, C₂—C₆ alkynyl, and C₃—C₆ cycloalkyl, |
| | each optionally substituted from 1 to 5 times with selected from the group |
| | consisting of H, halogen, —OH, and —NH₂, wherein R₁₁ is defined as below; |
| | R₁₁ is independently H, C₁—C₆ alkyl, C₂—C₆ alkenyl, C₂—C₆ alkynyl, C₃—C₆ |
| | cycloalkyl, or monocyclic aryl. |
| Family II | 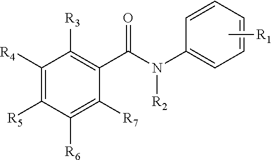 |
| | wherein: |
| | R₁—R₇ are independently H, halogen, —OH, —OR₈, —NH₂, —NHR₈, —NR₈R₉, —CHO, |
| | —C(O)OH, —C(O)OR₈, —C(O)NHR₈, —C(O)NR₈R₉, —N(H)C(O)OR₈, C₁—C₆ alkyl, |
| | C₂—C₆ alkenyl, C₂—C₆ alkynyl, C₂—C₆ cycloalkyl, monocyclic aryl, arylalkyl, or a |
| | mono or bicyclic heterocycle containing 1 to 5 heteroatoms selected from the |
| | group consisting of oxygen, nitrogen, and sulfur, each one of R₁—R₇ optionally |
| | substituted from 1 to 5 times with substituents selected from the group |
| | consisting of H, halogen, —OH, —OR₁₀, —NH₂, —NHR₁₀, —NR₁₀R₁₁, C₁—C₆ alkyl, |
| | C₂—C₆ alkenyl, C₂—C₆ alkynyl, C₃—C₆ cycloalkyl, monocyclic aryl, and arylalkyl, |
| | wherein R₈—R₁₀ are defined as below; |
| | or R₄ and R₅ or R₅ and R₆ can combine to form a 3- to 6-membered carbocycle |
| | or heterocycle containing 1 to 5 heteroatoms selected from the group consisting |
| | of oxygen, nitrogen, and sulfur, wherein the carbocycle or heterocycle is |
| | optionally substituted 1 to 5 times with substituents selected from the group |
| | consisting of H, halogen, cyano, —OH, —NH₂, C₁—C₆ alkyl, C₂—C₆ alkenyl, C₂—C₆ |
| | alkynyl, C₃—C₆ cycloalkyl, monocyclic aryl, and arylalkyl; and |
| | R₈—R₁₀ are independently H, C₁—C₆ alkyl, C₂—C₆ alkenyl, C₂—C₆ alkynyl, C₃—C₆ |
| | cycloalkyl, monocyclic aryl, arylalkyl, or a mono or bicyclic heterocycle or |
| | heteroaryl containing 1 to 5 heteroatoms selected from the group consisting of |
| | oxygen, nitrogen, and sulfur, each one of R₈, R₁₀ optionally substituted from 1 |
| | to 5 times with substituents selected from the group consisting of H, halogen, |
| | cyano, —OH, —NH₂, C₁—C₆ alkyl, C₂—C₆ alkenyl, C₂—C₆ alkynyl, C₃—C₆ |
| | cycloalkyl, monocyclic aryl, arylalkyl, and a mono or bicyclic heterocycle or |
| | heteroaryl containing 1 to 5 heteroatoms selected from the group consisting of |
| | oxygen, nitrogen, and sulfur. |
| Family III | 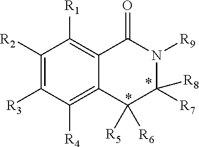 |
| | wherein: |
| | carbon atoms designated * are in the R or S configuration; and |
| | R₁—R₉ are independently H, halogen, —OH, —OR₁₀, —NH₂, —NHR₁₀, —NR₁₀R₁₁, |
| | —CHO, —C(O)OH, —C(O)OR₁₀, —C(O)NHR₁₀, —C(O)NR₁₀R₁₁, —N(H)C(O)OR₁₀, |
| | C₁—C₆ alkyl, C₂—C₆ alkenyl, C₂—C₆ alkynyl, C₃—C₆ cycloalkyl, monocyclic aryl, |
| | arylalkyl, or a mono or bicyclic heterocycle containing 1 to 5 heteroatoms |
| | selected from the group consisting of oxygen, nitrogen, and sulfur, each one of |
| | R₁—R₉ optionally substituted from 1 to 5 times with substituents selected from |
| | the group consisting of H, halogen, —OH, —C(O)OH, —CR₁₀R₁₁R₁₂, —OR₁₂, —NH₂, |
| | —NHR₁₂, —NR₁₂R₁₃, C₁—C₆ alkyl, C₂—C₆ alkenyl, C₂—C₆ alkynyl, C₃—C₆ |
| | cycloalkyl, monocyclic aryl, adamantyl, and arylalkyl, wherein R₁₀—R₁₃ are |
| | defined as below; |
| | R₁₀—R₁₃ are independently H, halogen, C₁—C₆ alkyl, C₂—C₆ alkenyl, C₂—C₆ |
| | alkynyl, C₃—C₆ cycloalkyl, monocyclic aryl, arylalkyl, or a mono or bicyclic |
| | heterocycle or heteroaryl containing 1 to 5 heteroatoms selected from the group |
| | consisting of oxygen, nitrogen, and sulfur, each one of R₁₀—R₁₃ optionally |
| | substituted from 1 to 5 times with substituents selected from the group |
| | consisting of H, halogen, cyano, —OH, —NH₂, C₁—C₆ alkyl, C₂—C₆ alkenyl, C₂—C₆ |
| | alkynyl, C₃—C₆ cycloalkyl, C₁—C₆ alkoxy, monocyclic aryl, arylalkyl, and a |
| | mono or bicyclic heterocycle or heteroaryl containing 1 to 5 heteroatoms |
| | selected from the group consisting of oxygen, nitrogen, and sulfur, wherein |
| | each one of R₁₀—R₁₃ substituents is optionally substituted from 1 to 5 times with |
| | substituents selected from the group H,—OH, —NH₂, —OCF₃, —NHR₁₄, —NR₁₄R₁₅, |
| | cyano, and nitro, wherein R₁₄ and R₁₅ are defined as below; |
| | R₁₄ and R₁₅ are independently H, C₁—C₆ alkyl, C₂—C₆ alkenyl, C₂—C₆ alkynyl, |
| | C₃—C₆ cycloalkyl, monocyelic aryl, arylalkyl, or a mono or bicyclic heterocycle |
| | or heteroaryl containing 1 to 5 heteroatoms selected from the group consisting |
| | of oxygen, nitrogen, and sulfur; or |
| | R₁₄ and R₁₅ can combine to form a 5- to 6-membered heterocycle or heteroaryl |
| | containing 1 to 5 heteroatoms selected from the group consisting of oxygen, |
| | nitrogen, and sulfur, each optionally substituted from 1 to 5 times with |
| | substituents selected from the group consisting H, —OH, —NH₂, cyano, and nitro. |
| Family IV | 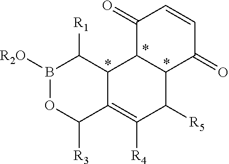 |
| | wherein: |
| | carbon atoms designated * are in the R or S configuration; and |
| | R₁—R₅ are independently H, halogen, —OH, —NH₂, —CHO, C₁—C₆ alkyl, C₂—C₆ |
| | alkenyl, C₂—C₆ alkynyl, C₃—C₆ cycloalkyl, monocyclic aryl, arylalkyl, or a mono |
| | or bicyclic heterocycle containing 1 to 5 heteroatoms selected from the group |
| | consisting of oxygen, nitrogen, and sulfur, each one of R₁-R₅ optionally |
| | substituted from 1 to 5 times with substituents selected from the group |
| | consisting of H, halogen, —OH, —OR₆, and —NH₂, wherein R₆ is defined as |
| | below; |
| | R₆ is H, —OH, —NH₂, C₁—C₆ alkyl, C₂—C₆ alkenyl, C₂—C₆ alkynyl, C₃—C₆ |
| | cycloalkyl, or monocyclic aryl. |
| Family V | 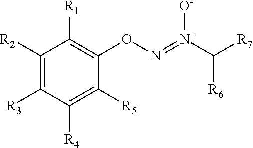 |
| | wherein: |
| | 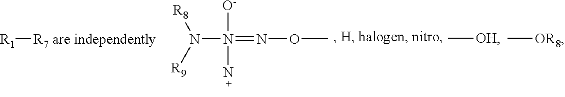 |
| | —OC(O)R₈, —OS(O)R₈, —OS(O)₂R₈, —NH₂, —NHR₈, —NR₈R₉, —CHO, —C(O)OH, |
| | —C(O)OR₈, —C(O)NHR₈, —C(O)NR₈R₉, —N(H)C(O)OR₈—, C₁—C₆ alkyl, C₂—C₆ |
| | alkenyl, C₂—C₆ alkynyl, C₃—C₆ cycloalkyl, monocyclic aryl, arylalkyl, or a mono |
| | or bicyclic heterocycle containing 1 to 5 heteroatoms selected from the group |
| | consisting of oxygen, nitrogen, and sulfur, each one of R₁—R₇ optionally |
| | substituted from 1 to 5 times with substituents selected from the group |
| | consisting of H, halogen, —OH, —OR₁₀, —NH₂, —NHR₁₀, —NR₁₀R₁₁, C₁—C₆ alkyl, |
| | C₂—C₆ alkenyl, C₂—C₆ alkynyl, C₃—C₆ cycloalkyl, monocyclic aryl, and arylalkyl, |
| | wherein R₈—R₁₁ are defined as below; |
| | or R₆ and R₇ can combine to form a 3- to 6-membered heterocycle or heteraryl |
| | containing 1 to 5 heteroatoms selected from the group consisting of oxygen, |
| | nitrogen, and sulfur, wherein each atoms of the heterocycle or heteroaryl is |
| | optionally substituted 1 to 5 times with substituents selected from the group |
| | consisting of H, halogen, —OH, cyano, —NH₂, and —N(H)C(O)OR₈, wherein R₈ |
| | is defined as below. |
| | R₈—R₁₁ are independently H, C₁—C₁₅ alkyl, C₂—C₁₅ alkenyl, C₂—C₁₅ alkynyl, |
| | C₃—C₆ cycloalkyl, monocyclic aryl, arylalkyl, or a mono or bicyclic heterocycle |
| | containing 1 to 5 heteroatoms selected from the group consisting of oxygen, |
| | nitrogen, and sulfur each optionally substituted with substituents selected from |
| | the group consisting of H, halogen, nitro, —OH, —OR₁₂, —OC(O)R₁₂, |
| | —NHC(O)R₁₂, wherein R₁₂ is defined below; and |
| | R₁₂ is independently H, C₁—C₁₅ alkyl, C₂—C₁₅ alkenyl, C₂—C₁₅ alkynyl, or C₃—C₆ |
| | cycloalkyl. |
| Family VI | 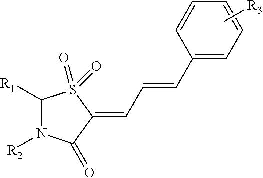 |
| | wherein: |
| | R₁—R₃ are independently H, halogen, —OH, —NH₂, C₁—C₆ alkyl, —NO₂, C₂—C₆ |
| | alkenyl, C₂—C₆ alkynyl, C₃—C₆ cycloalkyl, monocyclic aryl, arylalkyl, or a mono |
| | or bicyclic heterocycle containing 1 to 5 heteroatoms selected from the group |
| | consisting of oxygen, nitrogen, and sulfur, each one of R₁—R₃ optionally |
| | substituted from 1 to 5 times with substituents selected from the group |
| | consisting of H, halogen, —OH, —OR₄, —NH₂, —NHR₄, —NR₄R₅, —NO₂, C₁—C₆ |
| | alkyl, C₂—C₆ alkenyl, C₂—C₆ alkynyl, C₃—C₆ cycloalkyl, monocyclic aryl, and |
| | arylalkyl, wherein R₄ and R₅ are defined as below; |
| | R₄ and R₅ are independently H, C₁—C₆ alkyl, C₂—C₆ alkenyl, C₂—C₆ alkynyl, |
| | C₃—C₆ cycloalkyl, monocyclic aryl, arylalkyl, or a mono or bicyclic heterocycle or |
| | heteroaryl containing 1 to 5 heteroatoms selected from the group consisting of |
| | oxygen, nitrogen, and sulfur, each one of R₄ and R₅ optionally substituted from |
| | 1 to 5 times with substituents selected from the group consisting of H, halogen, |
| | cyano, —OH, —NH₂, C₁—C₆ alkyl, C₂—C₆ alkenyl, C₂—C₆ alkynyl, C₃—C₆ |
| | cycloalkyl, monocyclic aryl, arylalkyl, and a mono or bicyclic heterocycle or |
| | heteroaryl containing 1 to 5 heteroatoms selected from the group consisting of |
| | oxygen, nitrogen, and sulfur. |
| Family VII | 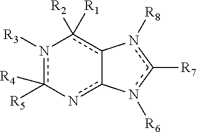 |
| | wherein: |
| | 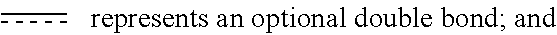 |
| | R₁—R₈ are independently H, halogen, —OH, —NH₂, C₁—C₆ alkyl, C₂—C₆ alkenyl, |
| | C₂—C₆ alkynyl, C₃—C₆ cycloalkyl, monocyclic aryl, arylalkyl, or a mono or |
| | bicyclic heterocycle containing 1 to 5 heteroatoms selected from the group |
| | consisting of oxygen, nitrogen, and sulfur, each one of R₁—R₈ optionally |
| | substituted from 1 to 5 times with substituents selected from the group |
| | consisting of H, halogen, —OH, —NH₂, C₁—C₆ alkyl, C₂—C₆ alkenyl, C₂—C₆ |
| | alkynyl, C₃—C₆ cycloalkyl, monocyclic aryl, and arylalkyl, wherein each of |
| | R₁—R₈ substituents is optionally substituted from 1 to 5 times with substituents |
| | selected from the group consisting of H, —OH, —NH₂, halogen, cyano, C₁—C₆ |
| | alkyl, C₂—C₆ alkenyl, C₂—C₆ alkynyl, C₃—C₆ cycloalkyl, monocyclic aryl, and |
| | arylalkyl; |
| | or R₁ and R₂ or R₄ and R₅ can combine to form (═O) or (═S); |
| Family VIII | 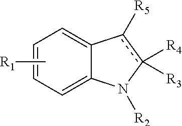 |
| | wherein: |
| | 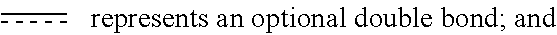 |
| | R₁—R₅ are independently H, halogen, —OH, —OR₆, —CHR₆, —NH₂, C₁—C₈ alkyl, |
| | methynyl, C₂—C₈ alkenyl, C₂—C₈ alkynyl, C₁—C₈ alkylcarboxyl, C₃—C₆ |
| | cycloalkyl, monocyclic aryl, arylalkyl, or a mono or bicyclic heterocycle or |
| | heteroaryl containing 1 to 5 heteroatoms selected from the group consisting of |
| | oxygen, nitrogen, and sulfur, each one of R₁—R₅ optionally substituted from 1 to |
| | 5 times with substituents selected from the group consisting of H, halogen, |
| | —OH, —NH₂, C₁—C₆ alkyl, C₂—C₆ alkenyl, C₂—C₆ alkynyl, C₃—C₆ cycloalkyl, |
| | monocyclic aryl, and arylalkyl, wherein R₆ is defined as below; |
| | or R₃ and R₄ can combine to form (═O), or (═S). |
| | R₆ is H, C₁—C₈ alkyl, C₂—C₈ alkenyl, C₂—C₈ alkynyl, C₃—C₆ cycloalkyl, |
| | monocyclic aryl, arylalkyl, or a mono or bicyclic heterocycle containing 1 to 5 |
| | heteroatoms selected from the group consisting of oxygen, nitrogen, and sulfur. |
| Family IX | 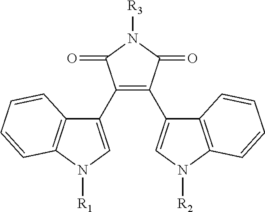 |
| | wherein: |
| | R¹—R³ are independently hydrogen, C₁—C₁₂ alkyl, or C₃—C₇ heterocycle, each |
| | optionally substituted from 1 to 3 times with substituents selected from the |
| | group consisting of pyrollidine, C₁—C₁₂ monoalkylamino, C₁—C₁₂ dialkylamino, |
| | and C₃—C₇ heterocycle containing 1 to 5 heteroatoms selected from the group |
| | consisting of oxygen, nitrogen, and sulfur, each one of R¹—R³ substituents |
| | optionally substituted from 1 to 4 times with substituents selected from |
| | hydrogen and C₁—C₁₂ alkyl |
| Family X | 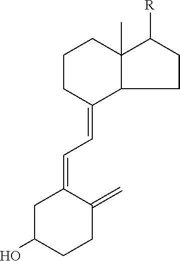 |
| | where R is a steroid homo side-chain defined by the structures I, II, III, IV, V |
| | and VI below: |
| | 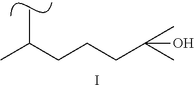 |
| | 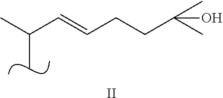 |
| | 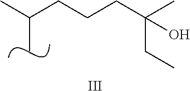 |
| | 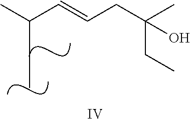 |
| | 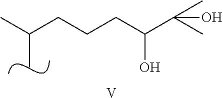 |
| | 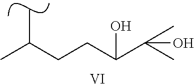 |
| Family XI | 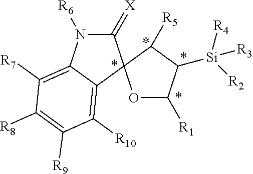 |
| | wherein: |
| | carbon atoms designated * are independently in the R or S configuration; arid |
| | X is optionally oxygen or sulfur; |
| | R₁—R₃ and R₅ are independently H, halogen, C₁—C₆ alkyl, C₂—C₆ alkenyl, C₂—C₆ |
| | alkynyl, C₃—C₆ cycloalkyl, C₄—C₇ cycloalkylalkyl, C₁—C₆ alkoxy, or C₁—C₆ |
| | amidoalkyl, each optionally substituted from 1 to 3 times with substituents |
| | selected from the group consisting of monocyclic aryl, monocyclic heteroaryl, |
| | arylalkyl, cyano, halogen, —NH₂, and hydroxy group, wherein the monocyclic |
| | aryl or heteroaryl containing 1-5 heteroatoms selected from the group |
| | consisting of nitrogen, sulfur, and oxygen, is optionally substituted from 1 to 3 |
| | times with substituents selected from the group consisting of C₁—C₆ alkyl, C₂—C₆ |
| | alkenyl, C₂—C₆ alkynyl, C₃—C₆ cycloalkyl, C₄—C₇ cycloalkylalkyl, C₁—C₆ alkoxy, |
| | arylalkoxy, and arylalkyl, each optionally substituted with substituents selected |
| | from the group consisting of halogen, hydroxy, —NH₂, and cyano; |
| | R₄ is a monocyclic aryl optionally substituted with substituents selected from |
| | the group consisting of halogen, hydroxy, —NH₂, cyano, C₁—C₆ alkyl, C₂—C₆ |
| | alkenyl, C₂—C₆ alkynyl, C₃—C₆ cycloalkyl, C₄—C₇ cycloalkylalkyl, C₁—C₆ alkoxy, |
| | and C₁—C₆ alkoxyetheryl; |
| | R₅—R₇ are independently halogen, H, —C(O)N(R₁₁)(R₁₂), C₁—C₆ alkyl, C₂—C₆ |
| | alkenyl, C₂—C₆ alkynyl, C₃—C₆ cycloalkyl, C₄—C₇ cycloalkylalkyl, monocyclic |
| | aryl, or arylalkyl, each optionally substituted from 1 to 3 times with |
| | substituents selected from the group consisting of phenyl, cyclocarbamoyl, |
| | cyano, halogen, —NH₂, hydroxy, R₁₂C(O)N(R₁₁)—, and R₁₂NHC(O)O— group, |
| | wherein the phenyl group is optionally substituted with substituents selected |
| | from the group consisting of halogen, hydroxy, —NH₂, and cyano, wherein R₁₁ |
| | and R₁₂ are defined below; |
| | or R₆ and R₇ can combine to form a fused 5 or 6-membered monocyclic |
| | heterocycle or heteroaryl containing 1-5 heteroatoms selected from the group |
| | consisting of oxygen, nitrogen, and sulfur, optionally substituted with |
| | substituents selected from the group consisting of C₁—C₆ alkyl, C₂—C₆ alkenyl, |
| | C₂—C₆ alkynyl, C₃—C₆ cycloalkyl, C₄—C₇ cycloalkylalkyl, monocyclic aryl, and |
| | monocyclic heteroaryl, wherein the heteroaryl containing 1-5 heteroatoms |
| | selected from the group consisting of oxygen, nitrogen, and sulfur, is optionally |
| | substituted with substituents selected from the group consisting of halogen, |
| | hydroxy, and cyano; |
| | R₈—R₁₀ are independently halogen, hydroxy, R₁₂NHC(O)O(R₅)—, |
| | R₁₂C(O)N(R₁₁)—, R₁₂NHC(O)O—,—NH₂, cyano, H, C₁—C₆ alkyl, C₂—C₆ alkenyl, |
| | C₂—C₆ alkynyl, C₃—C₆ cycloalkyl, C₄—C₇ cycloalkylalkyl, C₁—C₆ alkoxy, C₁—C₆ |
| | alkoxyetheryl, C₁—C₆ amidoalkyl, 4- to 6- membered lactam, or C₃—C₆ |
| | cyclocarbamoyl, each optionally substituted from 1 to 3 times with substituents |
| | selected from the group consisting of halogen, hydroxy, —NH_{2,} cyano, |
| | R₁₃NHC(O)O—, wherein R₁₂—R₁₃ are defined as below;. |
| | R₁₁—R₁₃ are independently H, C₁—C₆ alkyl, C₂—C₆ alkenyl, C₂—C₆ alkynyl, C₃—C₆ |
| | cycloalkyl, C₄—C₇ cycloalkylalkyl, C₁—C₆ alkoxy, 5- to 6- monocyclic aryl, or |
| | arylalkyl; each optionally substituted from 1 to 3 times with substituents |
| | selected from the group consisting of H, C₁—C₆ alkyl, C₂—C₆ alkenyl, C₂—C₆ |
| | alkynyl, C₃—C₆ cycloalkyl, C₄—C₇ cycloalkylalkyl, C₁—C₆ alkoxy, halogen, |
| | —C(O)NHOH, hydroxy, —NH₂, and cyano; |
| | or R₁₁ and R₁₂ can combine to form a mono or bicyclic heterocycle or |
| | heteroaryl containing 1 to 5 heteroatoms selected from the group consisting of |
| | oxygen, nitrogen, and sulfur. |
| Family XII | 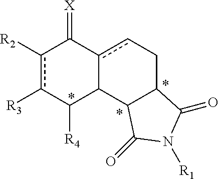 |
| | wherein: |
| | carbon atoms designated * are independently in the R or S configuration; and |
| | 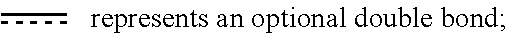 |
| | X is oxygen, sulfur, or NR₅, wherein R₅ is defined as below; |
| | R₁—R₄ are independently halogen, hydroxy, —NH₂, cyano, H, C₁—C₆ alkyl, C₂—C₆ |
| | alkenyl, C₂—C₆ alkynyl, C₃—C₆ cycloalkyl, C₄—C₇ cycloalkylalkyl, aryl alkyl, or |
| | mono or bicyclic heterocycle containing 1 to 5 heteroatoms selected from the |
| | group consisting of oxygen, nitrogen, and sulfur, —C(O)OCH₂CH₃, |
| | —(C₆H₅)O(C₆H₅), each optionally substituted from 1 to 3 times with substituents |
| | selected from the group consisting of C₁—C₆ alkyl, C₂—C₆ alkenyl, C₂—C₆ alkyl, |
| | C₃—C₆ cycloalkyl, monocyclic aryl, or mono or bicyclic heterocycle containing |
| | 1 to 5 heteroatoms selected from the group consisting of oxygen, nitrogen, and |
| | sulfur, cyano, halogen, —NH₂, R₅OC(O)—, OR₅, and hydroxy group, wherein said |
| | phenyl group is optionally substituted with substituents selected from the group |
| | consisting of halogen, hydroxy, —NH₂, and cyano; |
| | or R₂ and R₃ can combine to form a fused 3 to 6-membered monocyclic |
| | heterocycle containing 1-5 heteroatoms selected from the group consisting of |
| | oxygen, nitrogen, and sulfur, optionally substituted with substituents selected |
| | from the group consisting of H, C₁—C₆ alkyl, C₂—C₆ alkenyl, C₂—C₆ alkynyl, |
| | C₃—C₆ cycloalkyl, C₄—C₇ cycloalkylalkyl, monocyclic aryl, and monocyclic |
| | heteroaryl wherein the heteroaryl containing 1-5 heteroatoms selected from the |
| | group consisting of oxygen, nitrogen, and sulfur, is optionally substituted with |
| | substituents selected from the group consisting of halogen, hydroxy, and cyano; |
| | R₅ is independently H, C₁—C₆ alkyl, C₂—C₆ alkenyl, C₂—C₆ alkynyl, C₃—C₆ |
| | cycloalkyl, monocyclic aryl, R₆OC(O)NH—, OR₁OR₂ and —OR₆; |
| | R₆ is H, C₁—C₆ alkyl, C₂—C₆ alkenyl, C₂—C₆ alkynyl, C₃—C₆ cycloalkyl, |
| | monocyclic aryl, arylalkyl, or a mono or bicyclic heterocycle or heteroaryl |
| | containing 1 to 5 heteroatoms selected from the group consisting of oxygen, |
| | nitrogen, and sulfur, each optionally substituted with halogen, hydroxyl, cyano, |
| | —NH₂, C₁—C₂ alkyl, C₁—C₂ alkoxy, C₂—C₆ alkenyl, heteroarylalkoxy, arylalkoxy, |
| | and C₃—C₆ cycloalkyl, wherein the heteroaryl contains 1 to 5 heteroatoms |
| | selected from the group consisting of oxygen, nitrogen, and sulfur. |
| Family XIII | 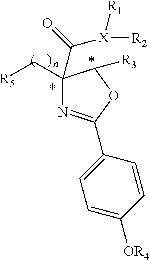 |
| | wherein: |
| | the carbon atom designated * is in the R or S configuration; and |
| | X is oxygen, nitrogen, or —CH—; |
| | n is an integer from 0 to 6; |
| | R₁ and R₂ are independently O, H, hydroxyl, C(O), —NH₂, C₁—C₆ alkyl, C₂—C₆ |
| | alkenyl, C₂—C₆ alkynyl, C₃—C₆ cycloalkyl, C₁—C₆ alkoxy, C₂—C₁₀ alkyletheryl, |
| | arylalkyl, or C₄—C₇ cycloalkylalkyl, each optionally substituted from 1 to 3 |
| | times with substituents selected from the group consisting of phenyl, cyano, |
| | halogen, N₃, —CH₂N₃, —NH₂, hydroxy, C₁—C₆ alkyl, C₂—C₆ alkenyl, C₂—C₆ |
| | alkynyl, C₃—C₆ cycloalkyl, and mono or bicyclic heteroaryl or heterocycle |
| | containing 1 to 5 heteroatoms selected from the group consisting of oxygen, |
| | nitrogen, and sulfur, wherein the phenyl group is optionally substituted from 1 |
| | to 3 times with substituents selected from the group consisting of N₃, —CH₂N₃, |
| | —NH₂, and cyano; |
| | or R₁ and R₂ can combine to form a 5- or 6-membered monocyclic heterocycle |
| | containing 1-5 heteroatoms selected from the group consisting of oxygen, |
| | nitrogen, and sulfur, optionally substituted from 1 to 4 times with substituents |
| | selected from the group consisting of H, C₁—C₆ alkyl, C₂—C₆ alkenyl, C₂—C₆ |
| | alkynyl, C₃—C₆ cycloalkyl, and C₄—C₇ cycloalkylalkyl; |
| | R₃ and R₅ are independently R₆OC(O)—, R₆SO₂—, R₆SO—, R₆S—, or 5- or 6- |
| | membered monocyclic aryl or heteroaryl optionally substituted from 1 to 3 |
| | times with substituents selected from the group consisting of H, N₃, —CH₂N₃, |
| | —NH₂, hydroxy, cyano, C₁—C₆ alkyl, C₂—C₆ alkenyl, C₂—C₆ alkynyl, C₃—C₆ |
| | cycloalkyl, and monocyclic aryl, wherein R₆ is defined as below; |
| | R₄ is independently H, C₁—C₆ alkyl, C₂—C₆ alkenyl, C₂—C₆ alkynyl, C₃—C₆ |
| | cycloalkyl, or C₄—C₇ cycloalkylalkyl, C₁—C₆ alkoxy, C₂—C₁₀ alkyletheryl, or |
| | arylalkyl, each optionally substituted from 1 to 3 times with substituents |
| | selected from the group consisting of phenyl, —OH, halogen, N₃, —CH₂N₃, —NH₂, |
| | cyano, and R₆NHC(O)O—, wherein R₆ is defined as below; |
| | R₆ is H, C₁—C₆ alkyl, C₂—C₆ alkenyl, C₂—C₆ alkynyl, C₃—C₆ alkynyl, C₃—C₆ |
| | cycloalkyl, mono cyclic aryl, arylalkyl or a mono or bicyclic heterocycle or |
| | heteroaryl containing 1 to 5 hereoatorns selected from the group consisting of |
| | oxygen, nitrogen, and sulfur, each optionally substituted with H, halogen, |
| | hydroxyl, cyano, —NH₂, and —C(O)NHOH; |
| | with the proviso that when X is oxygen, one of R₁ or R₂ is absent. |
| Family XIV | 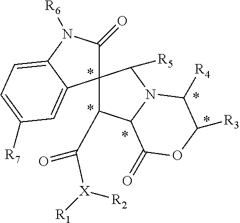 |
| | wherein: |
| | carbon atoms designated * are independently in the R or S configuration; and |
| | X is oxygen or nitrogen; |
| | R₁ and R₂ are independently H, C₁—C₆ alkyl, C₂—C₆ alkenyl, C₂—C₆ alkynyl, |
| | C₃—C₆ cycloalkyl, or C₄—C₇ cycloalkylalkyl, and arylalkyl, each optionally |
| | substituted from 1 to 3 times with substituents selected from the group |
| | consisting of phenyl, cyano, halogen, —NH₂, and hydroxy group, wherein the |
| | phenyl group is optionally substituted from 1 to 3 times with substituents |
| | selected from the group consisting of N₃, —CH₂N₃, —NH₂, and cyano; |
| | or R₁ and R₂ can combine to form a 3- to 10-membered monocyclic heterocycle |
| | containing 1-5 heteroatoms selected from the group consisting of oxygen, |
| | nitrogen, and sulfur, optionally substituted from 1 to 3 times with substituents |
| | selected from the group consisting of H, C₁—C₆ alkyl, C₂—C₆ alkenyl, C₂—C₆ |
| | alkynyl, C₃—C₆ cycloalkyl, C₄—C₇ cycloalkylalkyl, monocyclic aryl, and |
| | rnonocyclic heteroaryl wherein the monocyclie aryl or heteroaryl containing 1- |
| | 5 heteroatoms selected from the group consisting of oxygen, nitrogen, and |
| | sulfur, and each of the monocyclic aryl or heteroaryl is optionally substituted |
| | with substituents selected from the group consisting of halogen, hydroxy, NH₂, |
| | and cyano; |
| | R₃—R₅ are independently 5- or 6-membered monocyclic aryl or heteroaryl |
| | optionally substituted with substituents selected from the group consisting of |
| | hydroxy, halogen, —NH₂, cyano, C₁—C₆ alkyl, C₂—C₆ alkenyl, C₂—C₆ alkynyl, |
| | C₃—C₆ cycloalkyl, C₄—C₇ cycloalkylalkyl, C₁—C₆ alkoxycarbonyl, C₁—C₆ |
| | alkoxycarboxyl, C₁—C₆ alkoxycarbamoyl, and —OR₈ each optionally substituted |
| | from 1 to 3 times with substituents selected from the group consisting of C₁—C₆ |
| | alkyl, C₁—C₆ alkenyl, C₁—C₆ alkynyl, phenyl, and hydroxyphenyl, wherein R₈ is |
| | defined as below; |
| | R₆ is independently H, C₁—C₆, alkyl, C₂—C₆ alkenyl, C₂—C₆ alkynyl, C₃—C₆ |
| | cycloalkyl, C₄—C₇ cycloalkylalkyl, R₉O—(CH₂)ₙ—OC(O)—, or R₉—OC(O)—, wherein |
| | n is an integer from 1 to 6 R₉ is defined as below; |
| | R₇ is independently H, C₁—C₆ alkyl, C₂—C₆ alkenyl, C₂—C₆ alkynyl, C₃—C₆ |
| | cycloalkyl, or C₄—C₇ cycloalkylalkyl, each optionally substituted from 1 to 3 |
| | times with substituents selected from the group consisting of phenyl, C₃—C₁₀ |
| | cycloalkyl, C₄—C₁₁ cycloalkylalkyl, C₃—C₁₀ cycloalkenyl, C₄—C₁₁ |
| | cycloalkenylalkyl, each optionally substituted from 1 to 3 times with |
| | substituents selected from the group consisting of C₁—C₆ alkyl, C₂—C₆ alkenyl, |
| | C₂—C₆ alkynyl, C₃—C₁₀ cycloalkyl, C₄—C₁₁ cycloalkylalkyl, C₃—C₁₀ cycloalkenyl, |
| | C₃—C₁₀ cycloalkenylalkyl, C₁—C₆ alkoxy, C₁—C₆ alkoxyetheryl, C₁—C₆ |
| | alkoxycarbonyl, C₁—C₆ alkoxycarboxyl, —OH, halogen, —NH₂, and cyano; |
| | R₈ is independently H, C₁—C₆ alkyl, C₂—C₆ alkenyl, C₂—C₆ alkynyl, C₃—C₆ |
| | cycloalkyl, C₄—C₇ cycloalkylalkyl, C₁—C₆ alkoxy, C₁—C₆ alkoxy carboxyl, C₁—C₆ |
| | alkoxy carbamoyl, each optionally substituted 1 to 5 times with substituents |
| | selected from the group consisting of H, —OH, —NH₂, cyano, and C₁—C₆ alkyl; |
| | R₉ is independently H, C₁—C₆ alkyl, C₂—C₆ alkenyl, C₂—C₆ alkynyl, C₃—C₆ |
| | cycloalkyl, C₄—C₇ cycloalkylalkyl; |
| | with the proviso that when X is oxygen, one of R₁ or R₂ is absent. |
| Family XV | 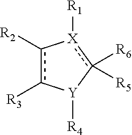 |
| | wherein: |
| | X and Y are independently C, O, N, or S; |
| | 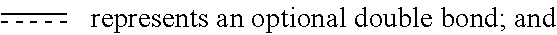 |
| | R₁—R₆ are independently H, halogen, —OH, —NH₂, C₁—C₆ alkyl, C₂—C₆ alkenyl, |
| | C₂—C₆ alkynyl, C₃—C₆ cycloalkyl, monocyclic aryl, arylalkyl, or a mono or |
| | bicyclic heterocycle containing 1 to 5 heteroatoms selected from the group |
| | consisting of oxygen, nitrogen, and sulfur, each of R₁—R₆ optionally substituted |
| | from 1 to 5 times with substituents selected from the group consisting of (═S), |
| | (═O), H, halogen, —OH, —NH₂, C₁—C₆ alkyl, C₂—C₆ alkenyl, C₂—C₆ alkynyl, C₃—C₆ |
| | cycloalkyl, C₁—C₆ alkoxy, monocyclic aryl, and arylalkyl; |
| | or R₅ and R₆ can combine to form (═O) or (═S). |
| Family XVI | 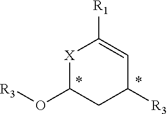 |
| | wherein: |
| | Carbon atoms designated * are in the R or S configuration; and |
| | X is O or S; |
| | R₁—R₃ are independently H, halogen, —OH, —OR₄, —NH₂, —NHR₄, —NR₄R₅, —CHO, |
| | —COOH, —COOR₄, —CONHR₄, —CONR₄R₅, —N(H)C(O)OR₄, C₁—C₆ alkyl, C₂—C₆ |
| | alkenyl, C₂—C₆ alkynyl, C₃—C₆ cycloalkyl, mono or polycyclic aryl, arylalkyl, or |
| | a mono or bicyclic heterocycle containing 1 to 5 heteroatoms selected from the |
| | group consisting of oxygen, nitrogen, and sulfur, each one of R₁—R₃ optionally |
| | substituted from 1 to 5 times with substituents selected from the group |
| | consisting of H, halogen, —OH, —OR₄, —NH₂, —NHR₄, —NR₄R₅, C₁—C₆ alkyl, |
| | C₂—C₆ alkenyl, C₂—C₆ alkynyl, C₃—C₆ cycloalkyl, mono or polycyclic aryl, and |
| | arylalkyl, each of R₁—R₃ substituents is optionally substituted from 1 to 5 times |
| | with substituents selected from H, C₁—C₆ alkyl, C₂—C₆ alkenyl, C₂—C₆ alkynyl, |
| | C₃—C₆ cycloalkyl, mono or polycyclic aryl, and arylalkyl, and or a mono or |
| | bicyclic heterocycle containing 1 to 5 heteroatoms selected from the group |
| | consisting of oxygen, nitrogen, and sulfur; wherein R₄ and R₅ are defined as |
| | below; |
| | R₄ and R₅ are independently H, halogen, —OH, —NH₂, C₁—C₆ alkyl, C₂—C₆ |
| | alkenyl, C₂—C₆ alkynyl, C₃—C₆ cycloalkyl, rnonocyclic aryl, arylalkyl, each |
| | optionally substituted from 1 to 3 times with substituents selected from the |
| | group consisting of H, halogen, —OH, and —NH₂; |
| | or R₄ and R₅ can combine to form 3- to 6-membered heterocycle or heteroaryl |
| | containing 1 to 5 heteroatoms selected from the group consisting of oxygen, |
| | nitrogen, and sulfur, each heterocycle or heteroaryl optionally substituted with |
| | substituents selected from the group consisting of H, C₁—C₆ alkyl, C₂—C₆ |
| | alkenyl, C₂—C₆ alkynyl, C₃—C₆ cycloalkyl, monocyclic aryl, arylalkyl, and or a |
| | mono or bicyclic heterocycle containing 1 to 5 heteroatoms selected from the |
| | group consisting of oxygen, nitrogen, and sulfur. |
| Family XVII | 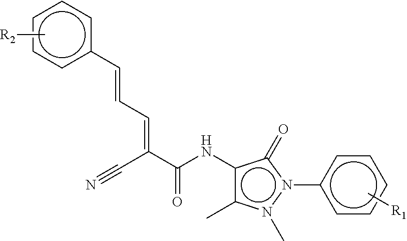 |
| | wherein: |
| | R₁ and R₂ are independently H, halogen, —OH, —NH₂, —NO₂, —CHO, —COOH, |
| | C₁—C₆ alkyl, C₂—C₆ alkenyl, C₂—C₆ alkynyl, C₃—C₆ cycloalkyl, monocyclic aryl, |
| | arylalkyl, or a mono or bicyclic heterocycle containing 1 to 5 heteroatoms |
| | selected from the group consisting of oxygen, nitrogen, and sulfur. |
| Family XVIII | 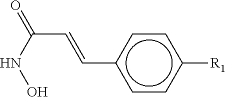 |
| | wherein: |
| | R₁ is independently H, —OH, —OR₂, —NH₂, —NHR₂, —NR₂R₃, —R₂NR₃, C₁—C₆ |
| | alkyl, C₁—C₆ alkoxy, C₂—C₆ alkenyl, C₂—C₆ alkynyl, C₃—C₆ cycloalkyl, |
| | monocyclic aryl, arylalkyl, or a mono or bicyclic heterocycle containing 1 to 5 |
| | heteroatoms selected from the group consisting of oxygen, nitrogen, and sulfur, |
| | each optionally substituted from 1 to 5 times with substituents selected from |
| | the group consisting of H, halogen, —OH, —NH₂, C₁—C₆ alkyl, C₂—C₆ alkenyl, |
| | C₂—C₆ alkynyl, C₃—C₆ cycloalkyl, monocyclic aryl, arylalkyl, and a mono or |
| | bicyclic heterocycle containing 1 to 5 heteroatoms selected from the group |
| | consisting of oxygen, nitrogen, and sulfur, wherein R₂ and R₃ are defined as |
| | below; |
| | R₂ and R₃ are independently H, C₁—C₆ alkyl, C₂—C₆ alkenyl, C₂—C₆ alkynyl, |
| | C₃—C₆ cycloalkyl, monocyclic aryl, arylalkyl, and a mono or bicyclic heterocycle |
| | containing 1 to 5 heteroatoms selected from the group consisting of oxygen, |
| | nitrogen, and sulfur. |
| Family XIX | 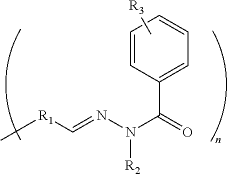 |
| | wherein |
| | n is an integer from 1 to 2; |
| | R₁—R₃ are independently H, —NH₂, —NO₂, —COOH, —C(O)NH₂, —C(O)NHOH, |
| | —OH, —OR₄, C₁—C₈ alkyl, C₂—C₈ alkenyl, C₂—C₈ alkynyl, C₃—C₈ cycloalkyl, C₄—C₉ |
| | cycloalkylalkyl, arylalkyl, mono or polycyclic aryl, a mono or bicyclic |
| | heterocycle, or heteroaryl containing 1 to 5 heteroatoms selected from the |
| | group consisting of oxygen, nitrogen, and sulfur, each of R₁—R₄ optionally |
| | substituted from 1 to 3 times with substituents selected from the group |
| | consisting of cyano, halogen, —NO₂, H, C₁—C₈ alkyl, C₁—C₈ alkoxy, aryloxy, |
| | C₂—C₈ alkenyl, C₂—C₈ alkynyl, C₃—C₈ cycloalkyl, C₄—C₉ cycloalkylalkyl, mono or |
| | polycyclic aryl, mono or bicyclic heterocycle or heteroaryl containing 1 to 5 |
| | heteroatoms selected from the group consisting of oxygen, nitrogen, and sulfur, |
| | N₃, —CH₂N₃, —NH₂, —COOH, —C(O)NH₂, —C(O)NHOH, R₄OC(O)N(R₅)—, and |
| | hydroxy group; |
| | R₄ and R₅ are H, C₁—C₈ alkyl, C₂—C₈ alkenyl, C₂—C₈ alkynyl, C₃—C₈ cycloalkyl, |
| | C₄—C₉ cycloalkylalkyl, arylalkyl, mono or polycyclic aryl, a mono or bicyclic |
| | heterocycle, or heteroaryl containing 1 to 5 heteroatoms selected from the |
| | group consisting of oxygen, nitrogen, and sulfur. |
| Family XX | 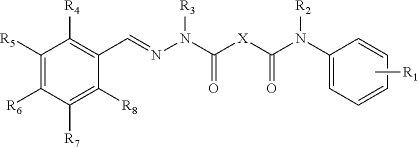 |
| | wherein: |
| | X is optionally C₁—C₆ alkyl, C₂—C₆ alkenyl, C₂—C₆ alkynyl, C₃—C₆ cycloalkyl, |
| | C₃—C₆ cycloalkenyl, 5- to 6- membered monocyclic aryl, or heteroaryl |
| | containing 1-5 heteroatoms selected from the group consisting of oxygen, |
| | sulfur, or nitrogen. |
| | R₁—R₈ are independently H, hydroxy, halogen, —NH₂, C₁—C₆ alkyl, C₂—C₆ |
| | alkenyl, C₂—C₆ alkynyl, C₃—C₆ cycloalkyl, C₃—C₆ cycloalkenyl, 5- to 6- |
| | membered monocyclic aryl, or heteroaryl containing 1-5 heteroatoms selected |
| | from the group consisting of oxygen, sulfur; |
| | or R₅ and R₆ can combine to form a mono carbocycle or heterocycle, wherein |
| | the heterocycle contains 1-5 heteroatoms selected from the group consisting of |
| | oxygen, sulfur, or nitrogen. |
| Family XXI | 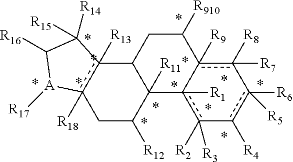 |
| | wherein: |
| | carbon atoms designated * are independently in the R or S configuration; and |
| | 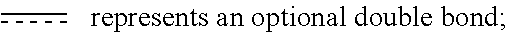 |
| | A is a CR₁₉ or O; |
| | R₁—R₁₈ are optionally and independently H, —OH, R₁₉C(O)—, R₁₉C(O)O—, —OR₁₉, |
| | halogen, C₁—C₆ alkyl, C₂—C₆ alkenyl, C₂—C₆ alkynyl, C₃—C₆ cycloalkyl, C₄—C₇ |
| | cycloalkylalkyl, C₁—C₁₀ alkoxycarboxyl, C₁—C₁₀ alkoxycarbamoyl, C₁—C₁₀ |
| | alkoxycarbonyl, aryloxy, arylalkoxy, or C₁—C₁₀ hydroxyketoalkyl, each |
| | optionally substituted from 1 to 3 times with substituents selected from the |
| | group consisting of hydroxy, —NH₂, cyano, halogen, and a mono or bicyclic |
| | heterocycle, or heteroaryl each containing 1 to 5 heteroatoms selected from the |
| | group consisting of oxygen, nitrogen, and sulfur, each one of R₁—R₁₈ |
| | substituents optionally substituted from 1 to 3 times with substituents selected |
| | from the group consisting of H, —OH, halogen, (═O), (═S), C₁—C₆ alkyl, C₂—C₆ |
| | alkenyl, C₂—C₆ alkynyl, C₃—C₆ cycloalkyl, C₄—C₇ cycloalkylalkyl, C₁—C₁₀ |
| | alkoxycarboxyl, C₁—C₁₀ alkoxycarbamoyl, C₁—C₁₀ alkoxycarbonyl, aryloxy, and |
| | arylalkoxy, wherein R₁₉ is defined as below; |
| | or R₂ and R₃ can combine to form a carbonyl group; |
| | R₆ and R₆ can combine to form the carbonyl group; |
| | R₇ and R₈ can combine to form the carbonyl group; |
| | R₁₄ and R₁₅ can combine to form the carbonyl group; |
| | R₅ and R₆, R₇ and R₈, R₆ and R₉ and R₁₅ and R₁₆ can combine to form |
| | independently a 3- to 6-membered cycloalkyl, or heterocycle containing 1 to 5 |
| | heteroatoms selected from the group consisting of nitrogen, sulfur, and |
| | oxygen, wherein said heterocycle is optionally substituted with substituents |
| | selected from the group consisting of H, —OH, halogen, C₁—C₆ alkyl, C₂—C₆ |
| | alkenyl, C₂—C₆ alkynyl, C₃—C₆ cycloalkyl, C₄—C₇ cycloalkylalkyl; |
| | R₁₉ is independently H, C₁—C₆ alkyl, C₂—C₆ alkenyl, C₂—C₆ alkynyl, C₃—C₆ |
| | cycloalkyl, C₄—C₇ cycloalkylalkyl, C₁—C₁₀ alkoxycarboxyl, C₁—C₁₀ |
| | alkoxycarbamoyl, C₁—C₁₀ alkoxycarbonyl, aryloxy, arylalkoxy, or C₁—C₁₀ |
| | hydroxyketoalkyl, each optionally substituted from 1 to 3 times with |
| | substituents selected from the group consisting of hydroxy, —NH₂, cyano, and |
| | halogen. |
| | with the proviso that when A is oxygen, R₁₆ is absent. |

Suitable compounds of Family I have the following structures:

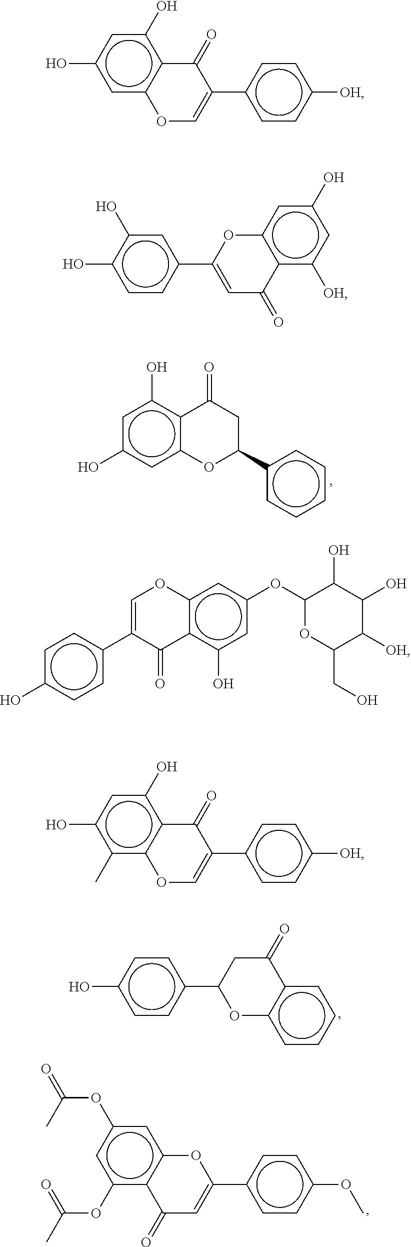

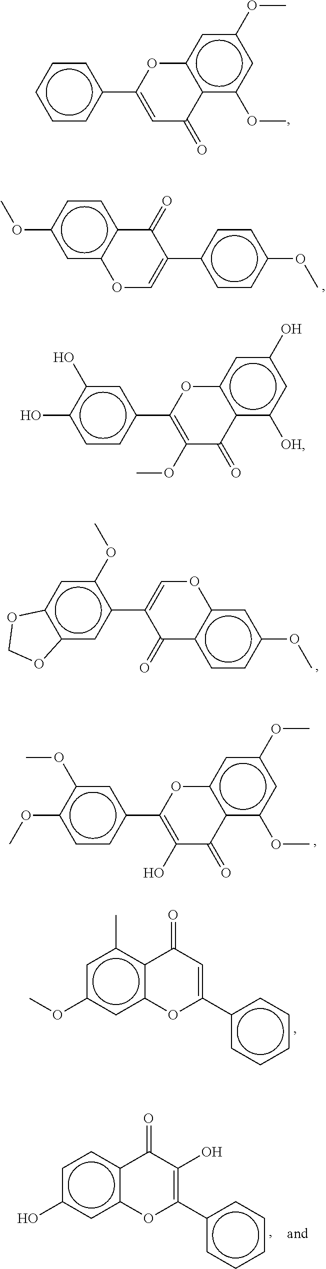

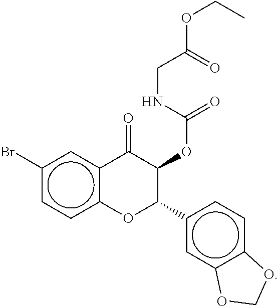

Suitable compounds of Family II have the following structures:

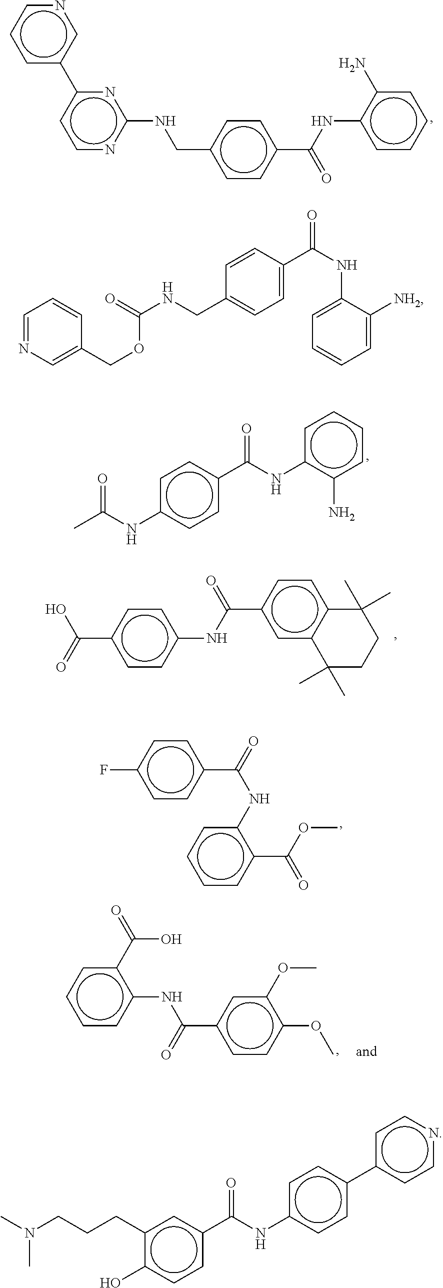

Suitable compounds of Family III have the following structures:

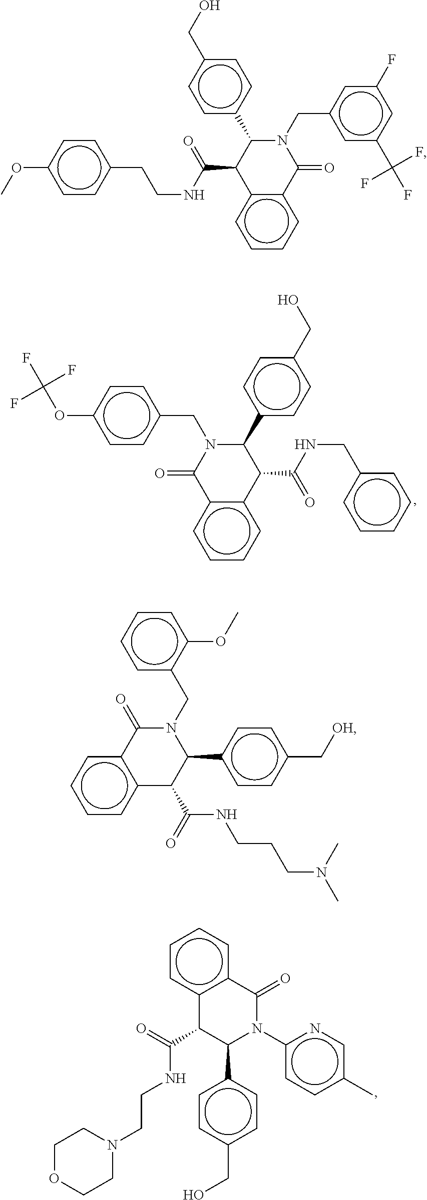

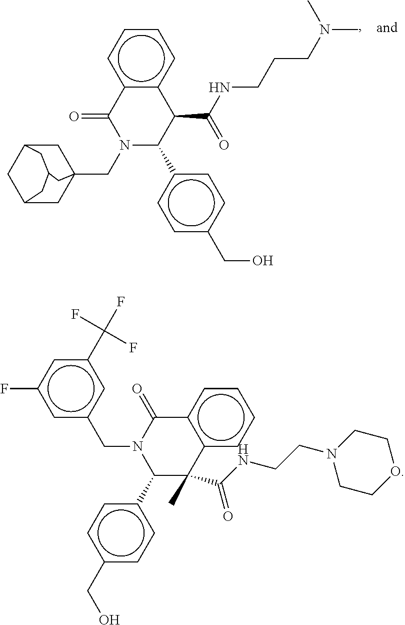

Example of suitable compounds of Family IV have the following structures:

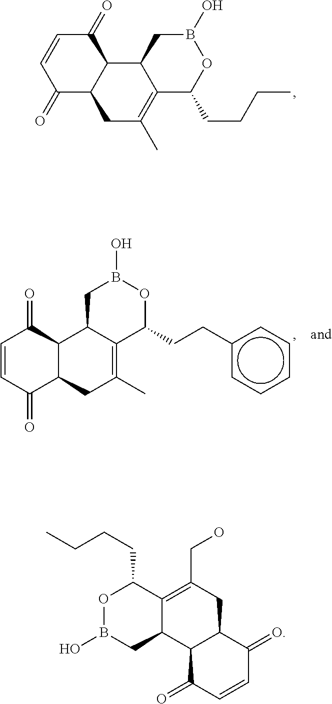

Suitable compounds of Family V have the following structures:

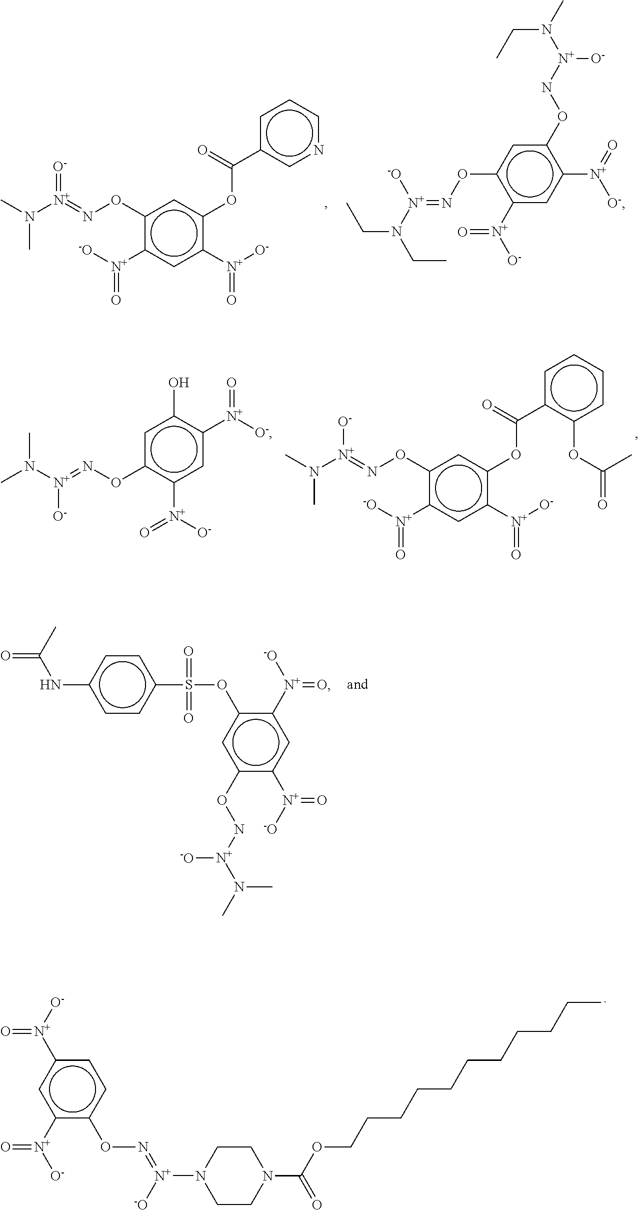

Suitable compounds of Family VI have the following structures:

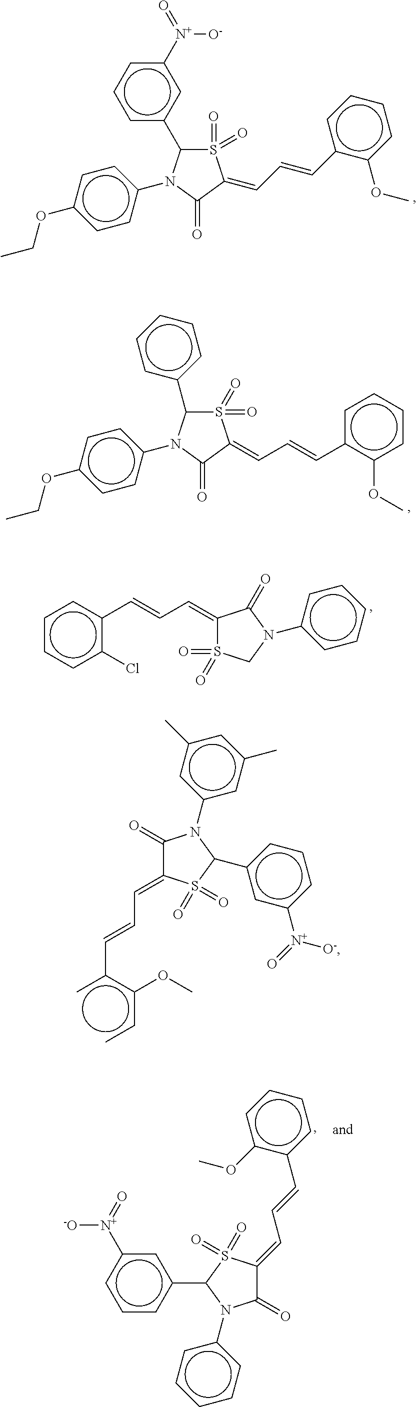

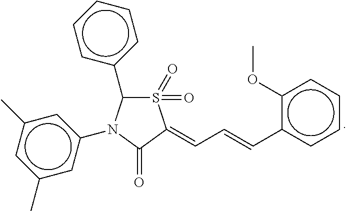

Suitable compounds of Family VII have the following structures:

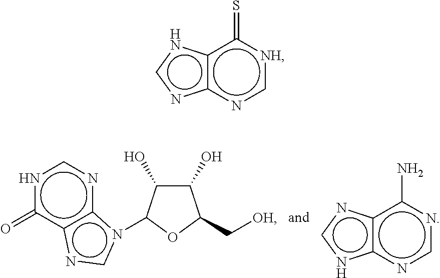

Suitable compounds of Family VIII have the following structures:

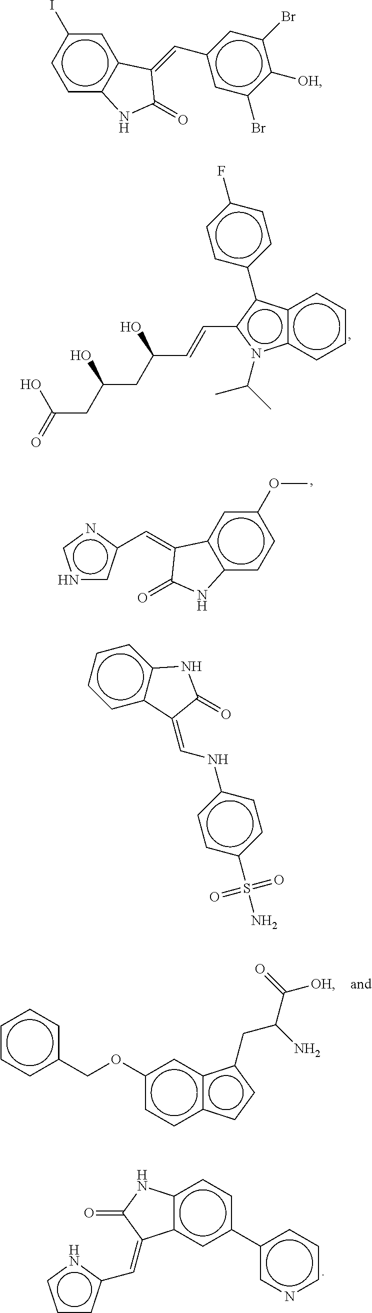

Suitable compounds of Family IX have the following structures:

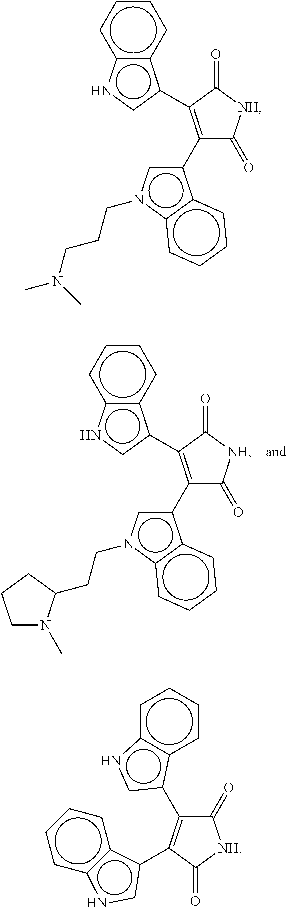

Suitable compounds of Family X have the following structures:

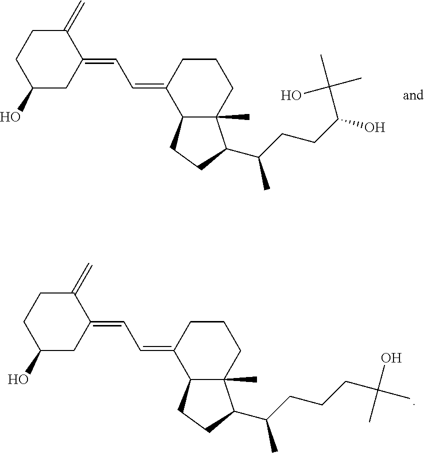 [SMILES: C=C1CC[C@H](O)C/C1=C\C=C1/CCC[C@]2(C)[C@@H]([C@H](C)CC[C@@H](O)C(C)(C)O)CC[C@@H]12 ; C=C1CC[C@H](O)C/C1=C\C=C1/CCC[C@]2(C)[C@@H]([C@H](C)CCCC(C)(C)O)CC[C@@H]12]

Suitable compounds of Family XI have the following structures:

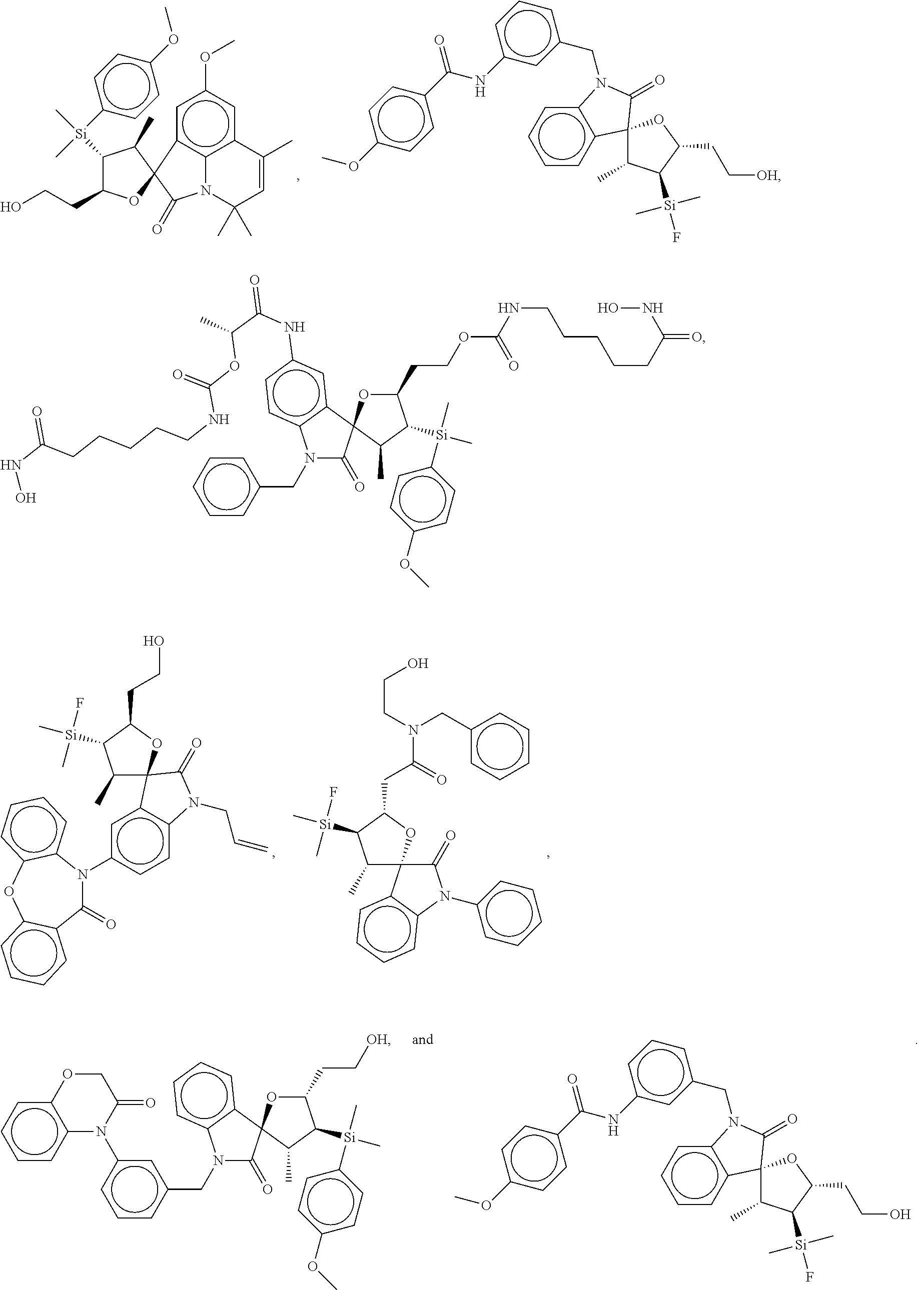

Suitable compounds of Family XII have the following structures:

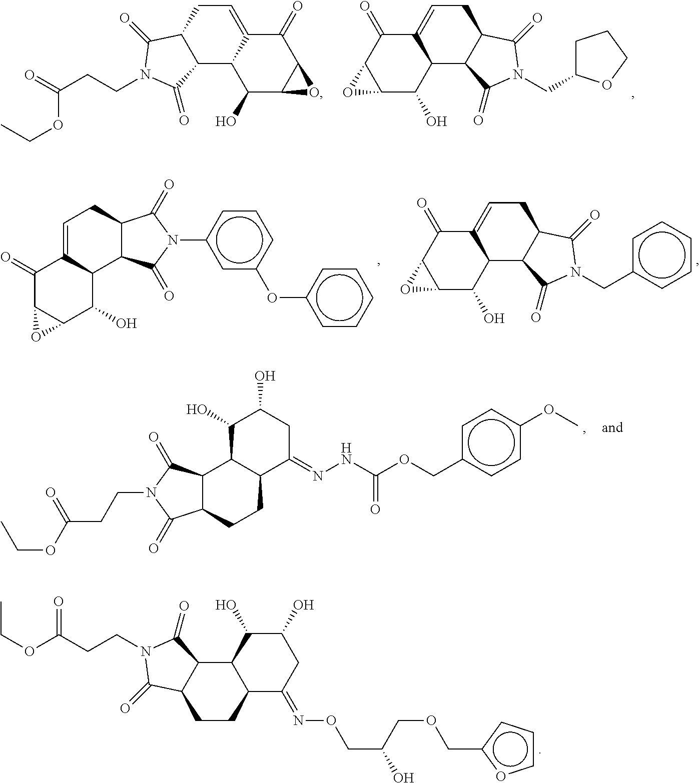

Suitable compounds of Family XIII have the following structures:

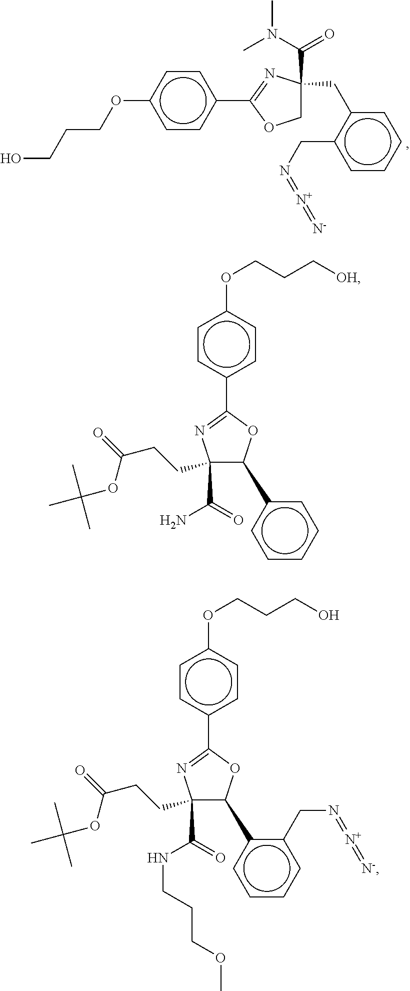

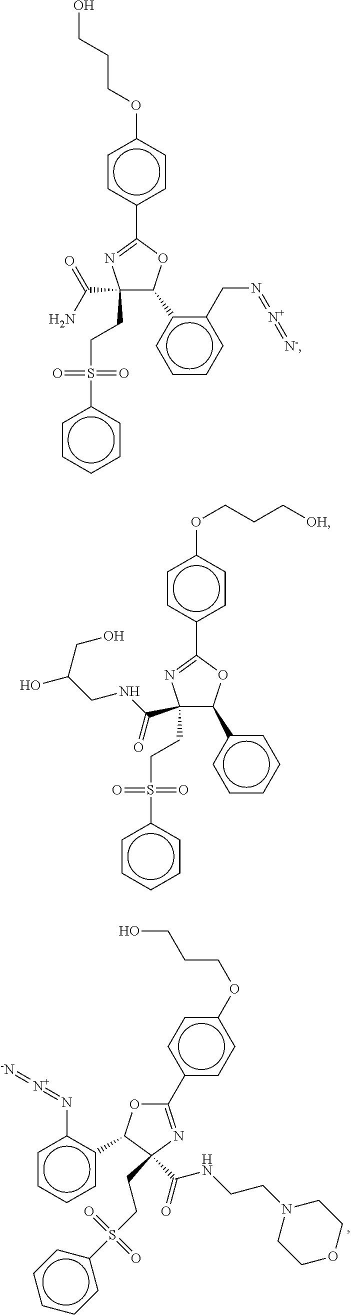

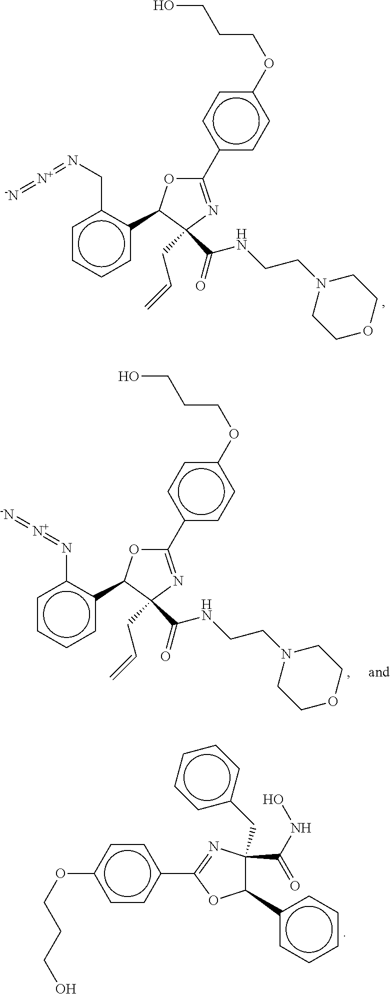

Suitable compounds of Family XIV have the following structures:

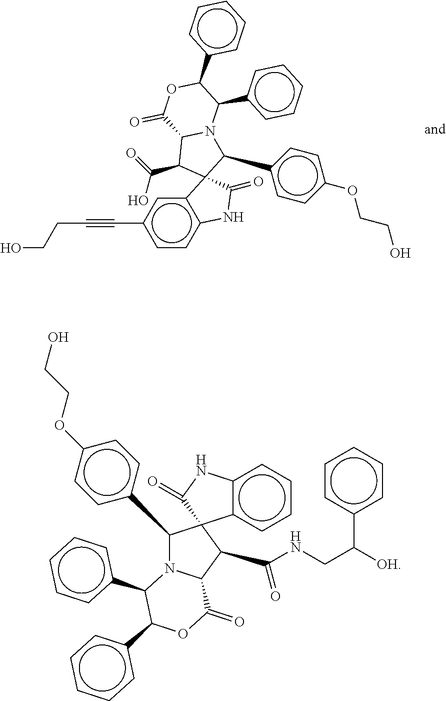

Suitable compounds of Family XV have the following structures:

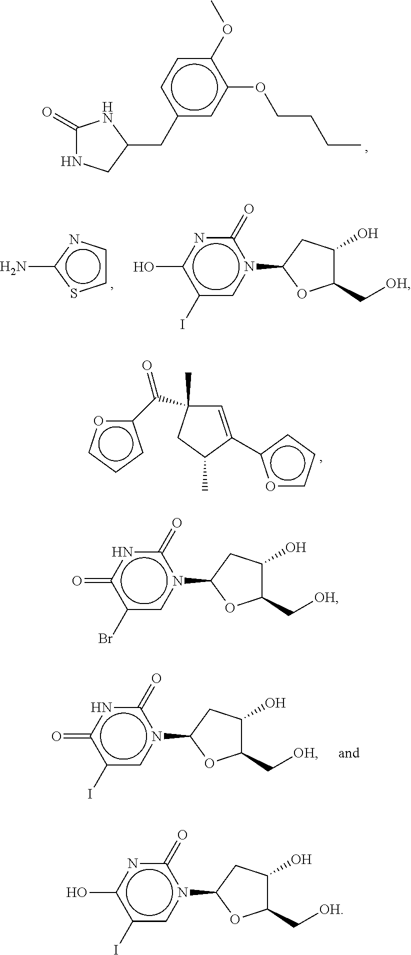

Suitable compounds of Family XVI have the following structures:

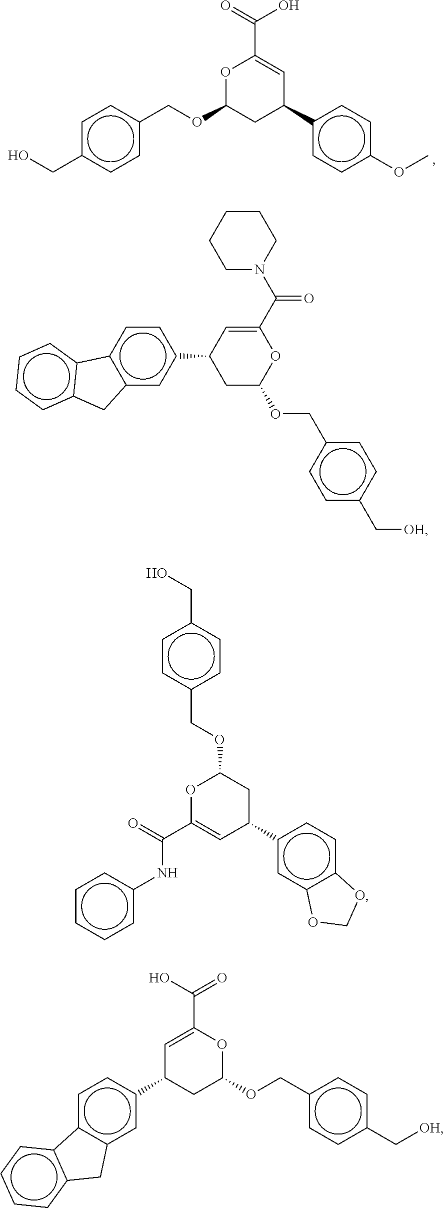

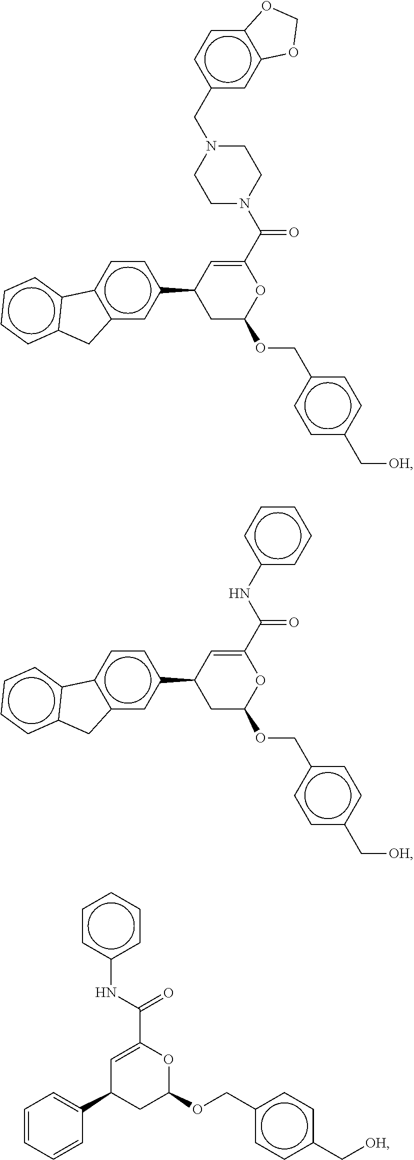

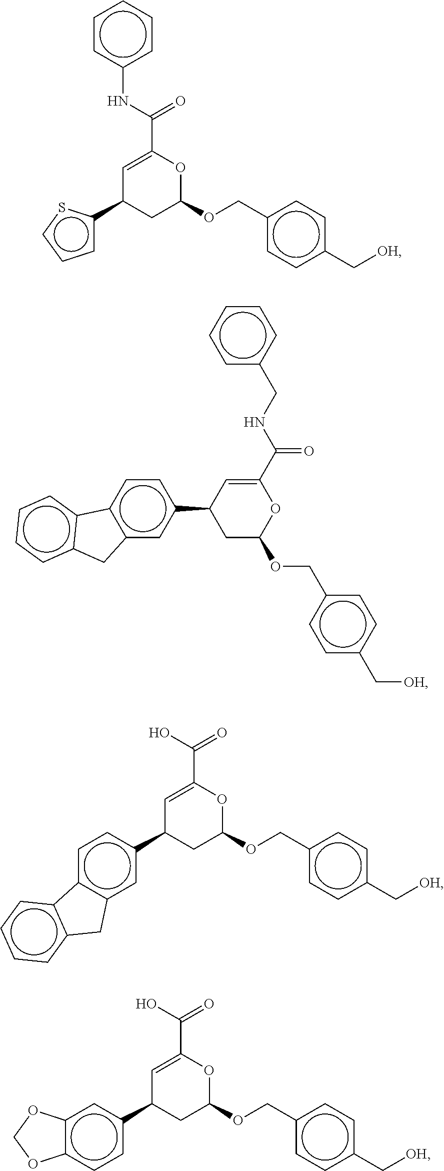

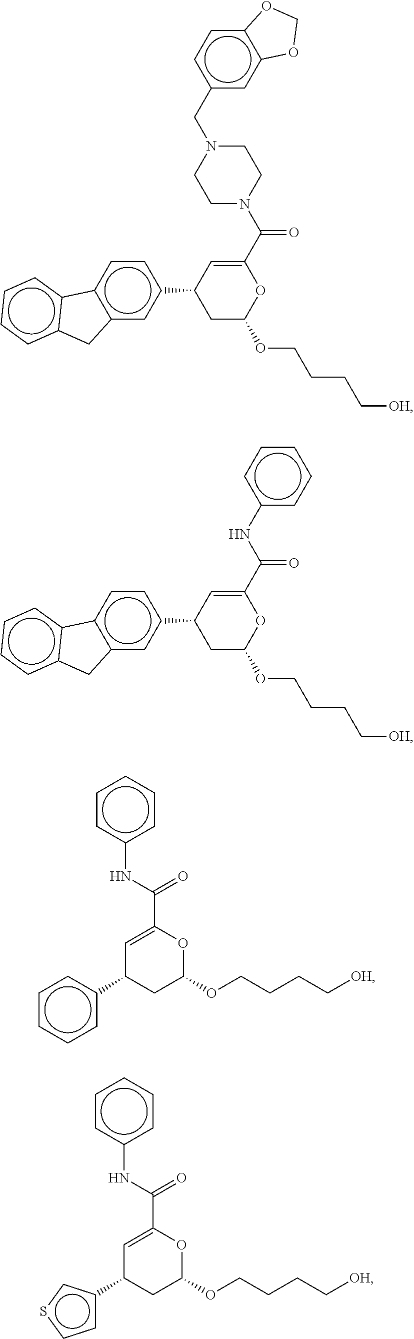

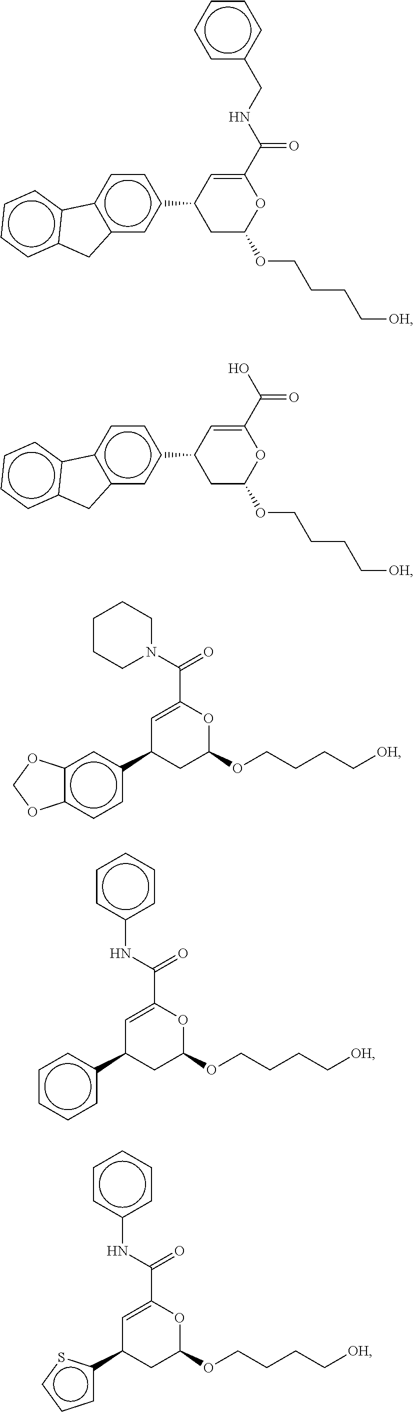

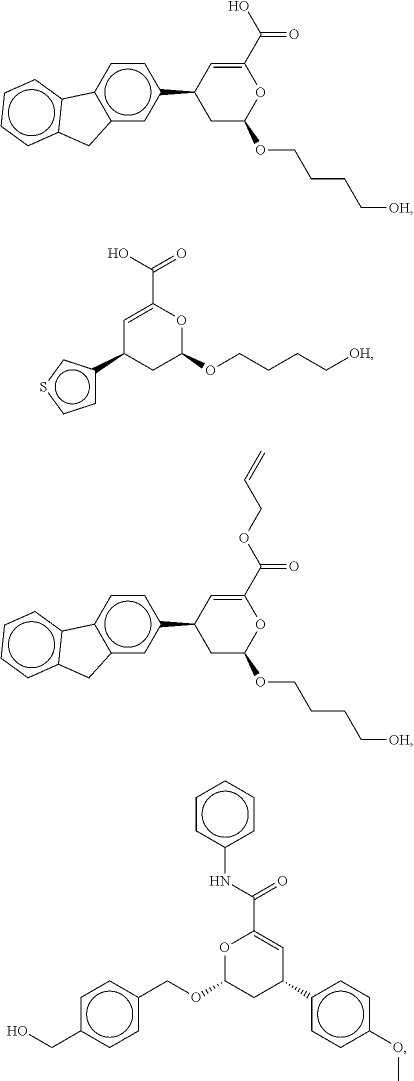

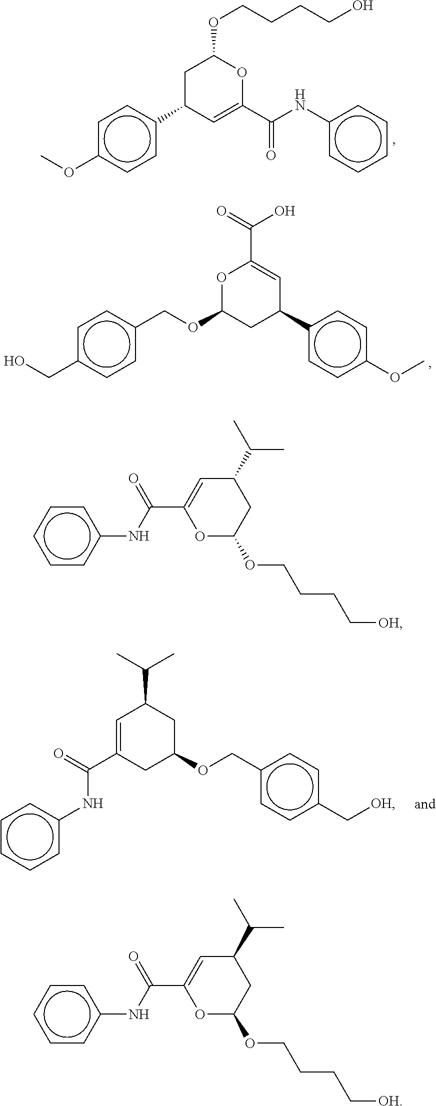

Suitable compounds of Family XVII have the following structures:

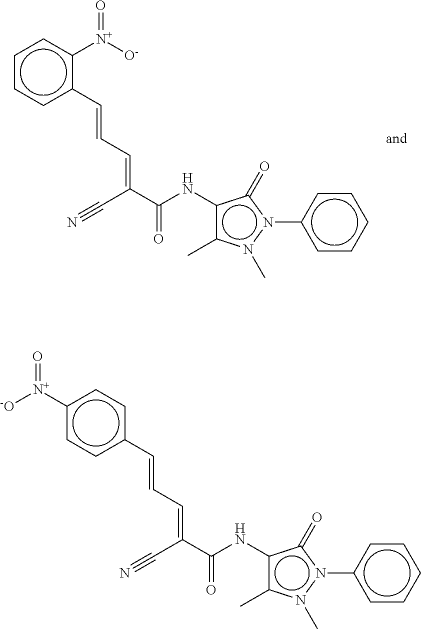

Suitable compounds of Family XVIII have the following structures:

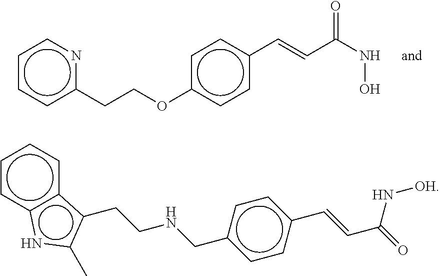

Suitable compounds of Family XIX have the following structures:

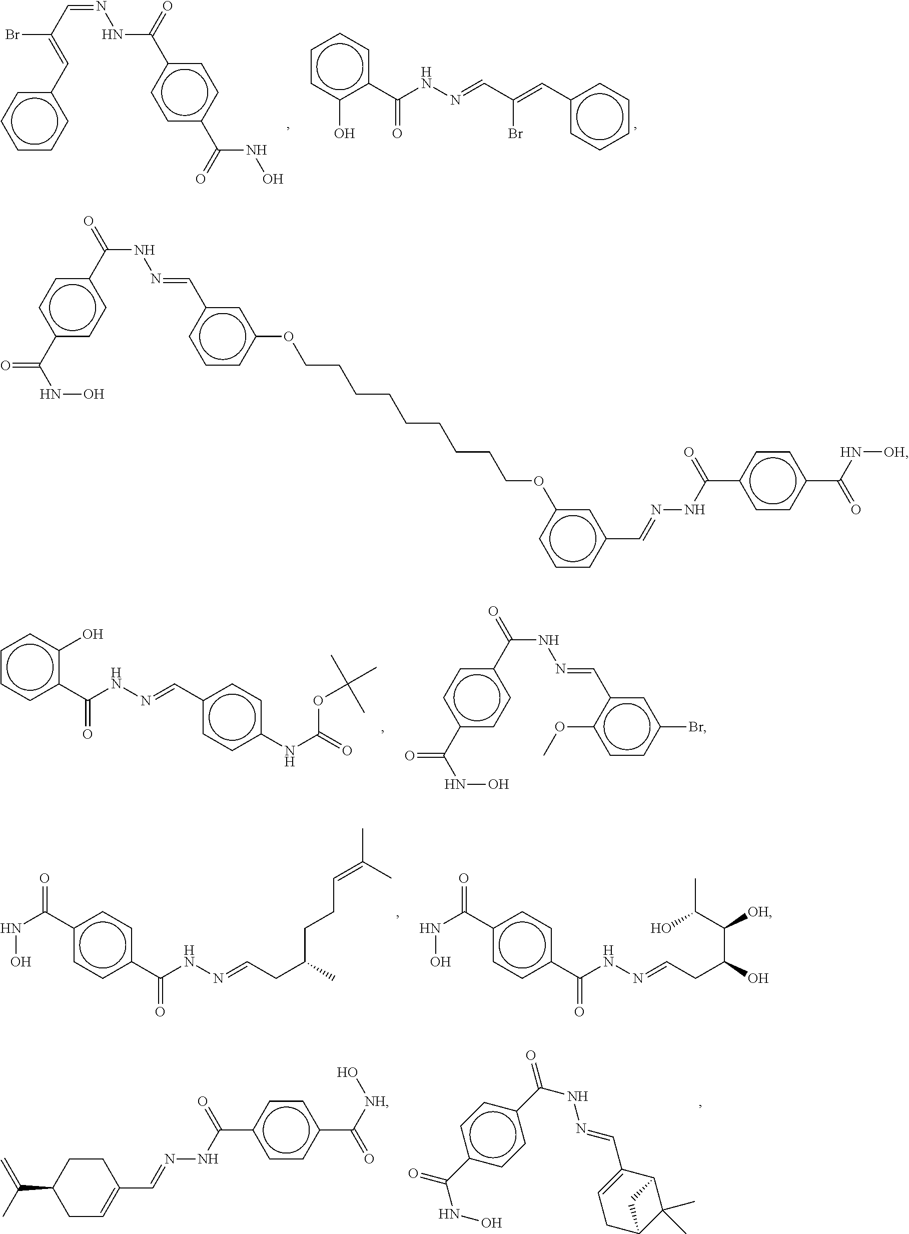

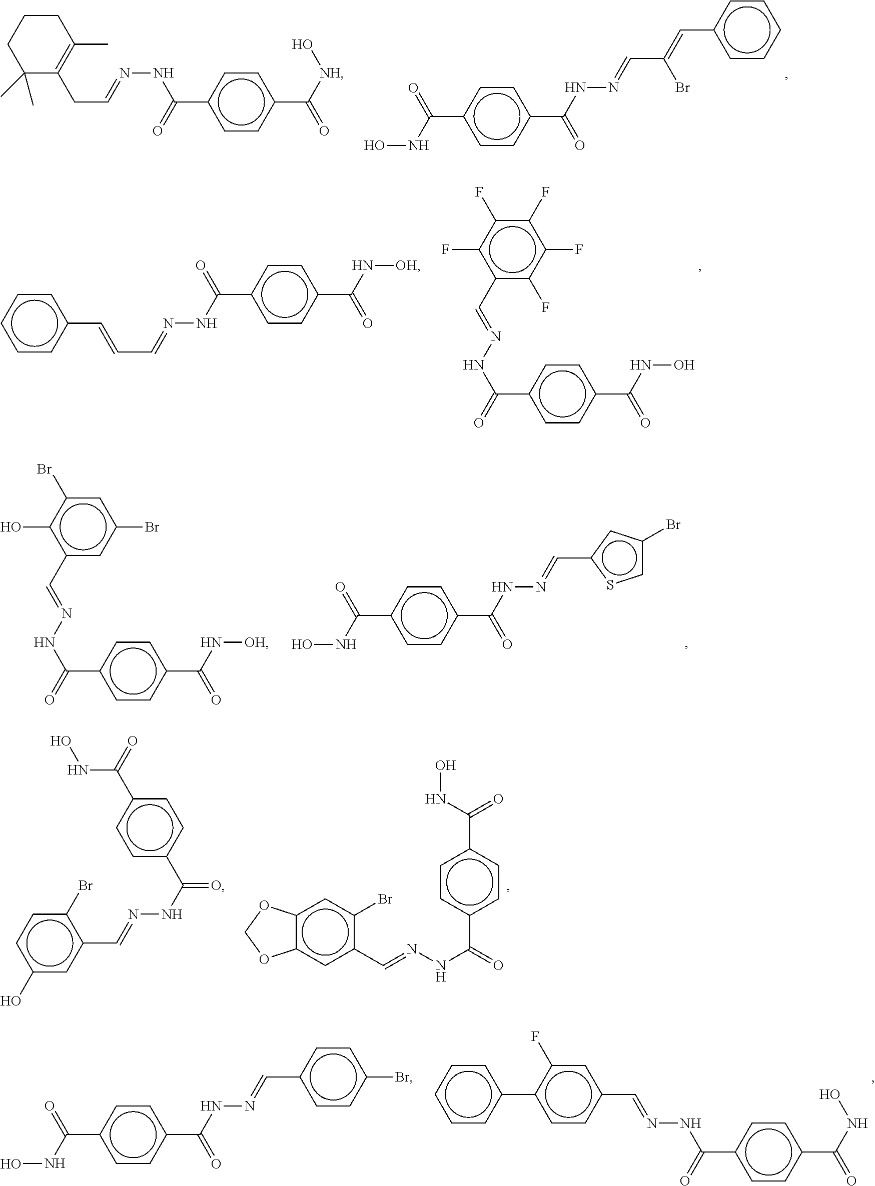

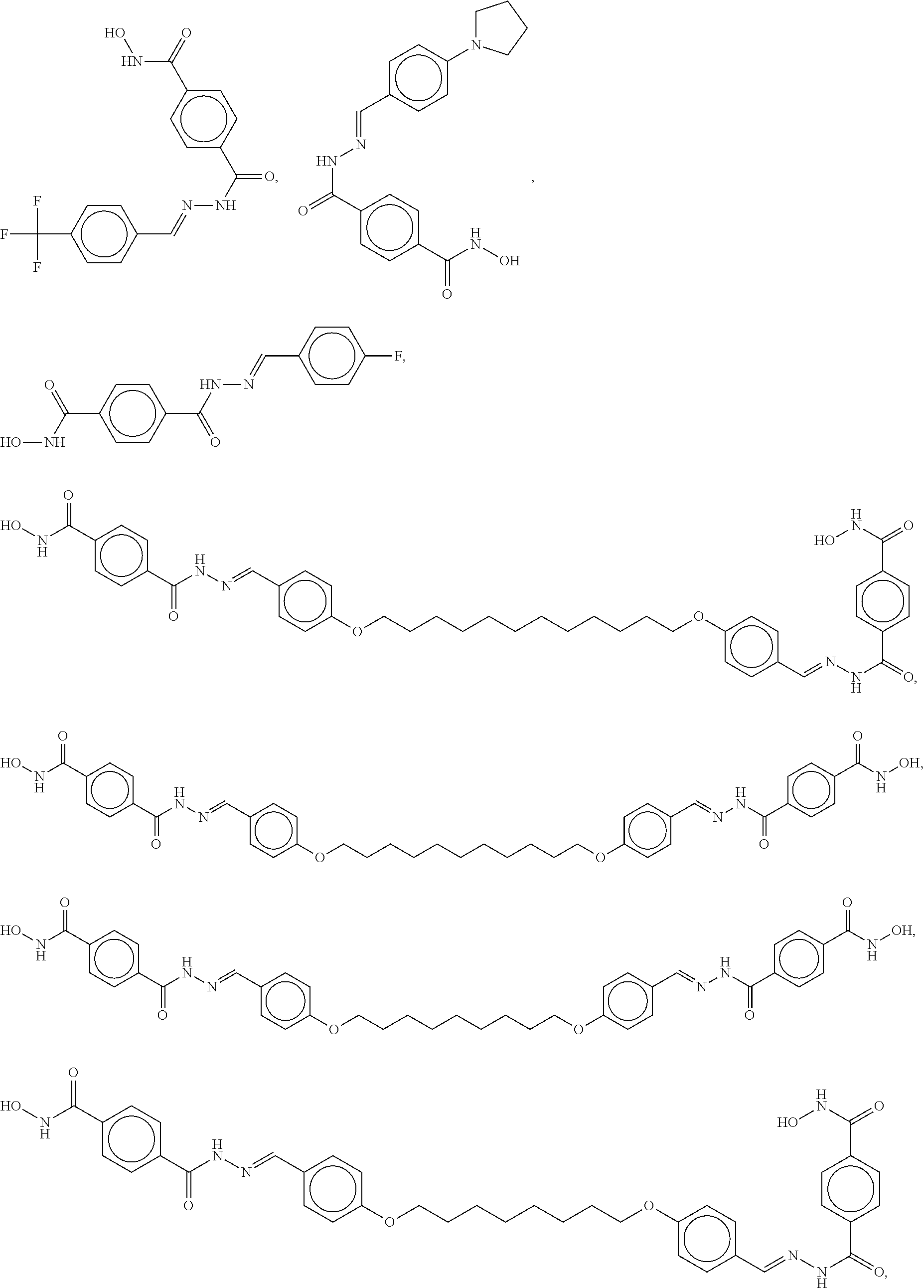

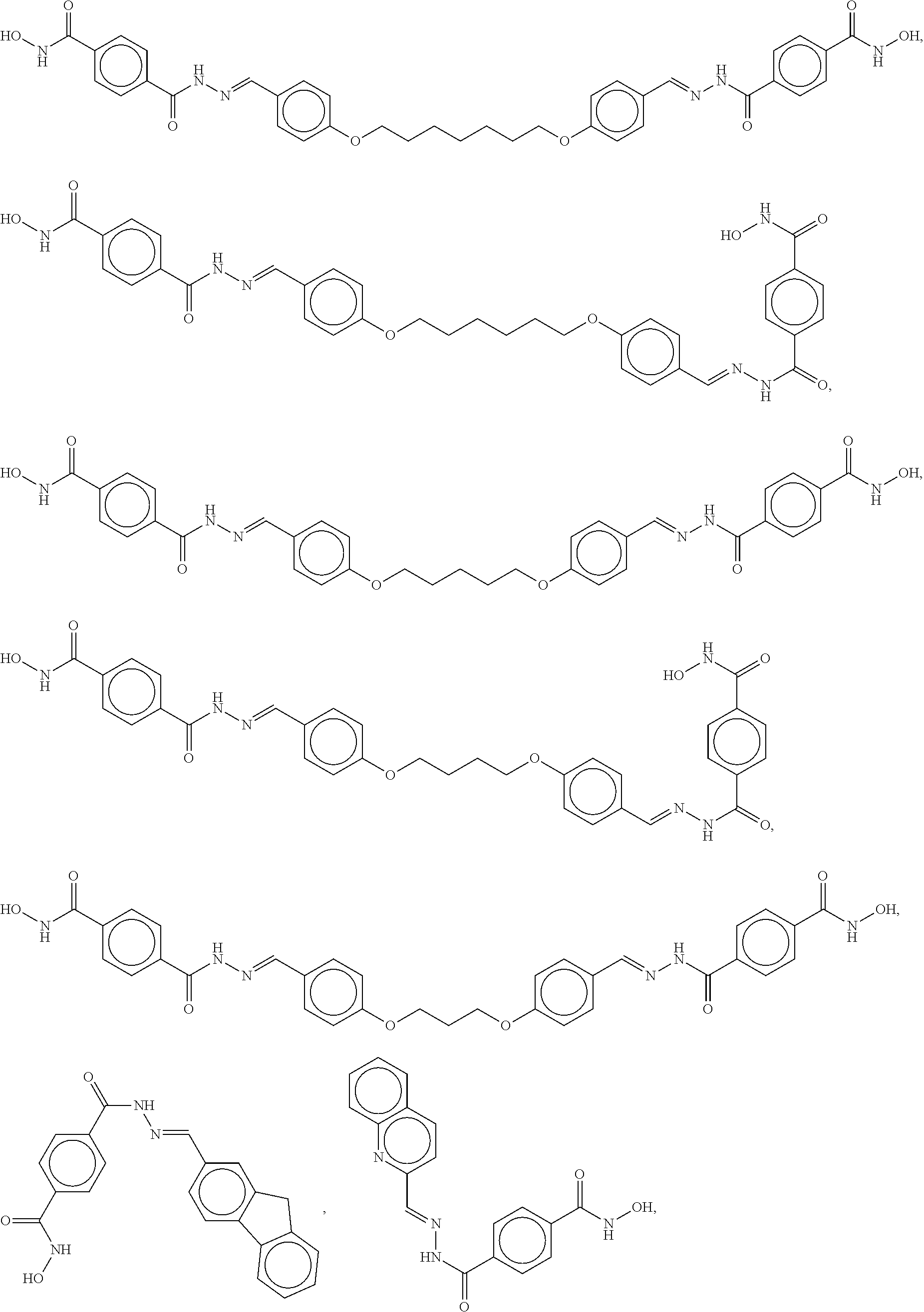

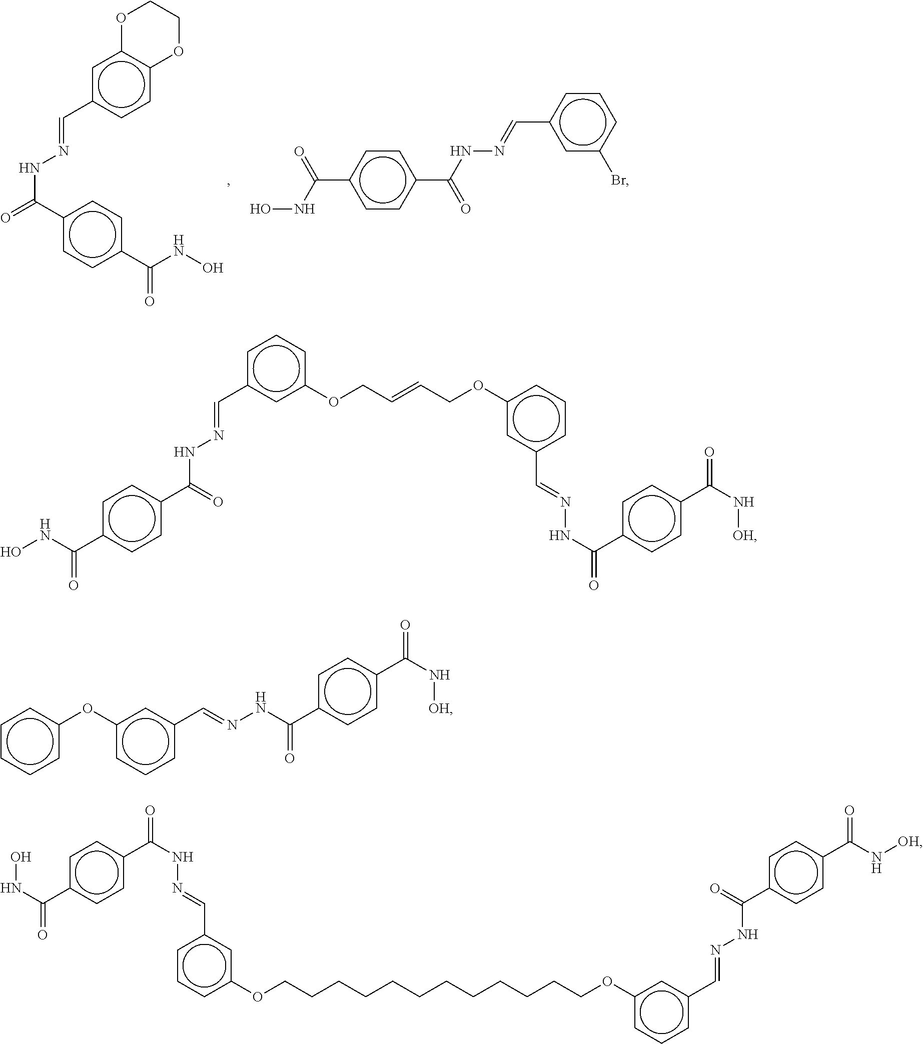

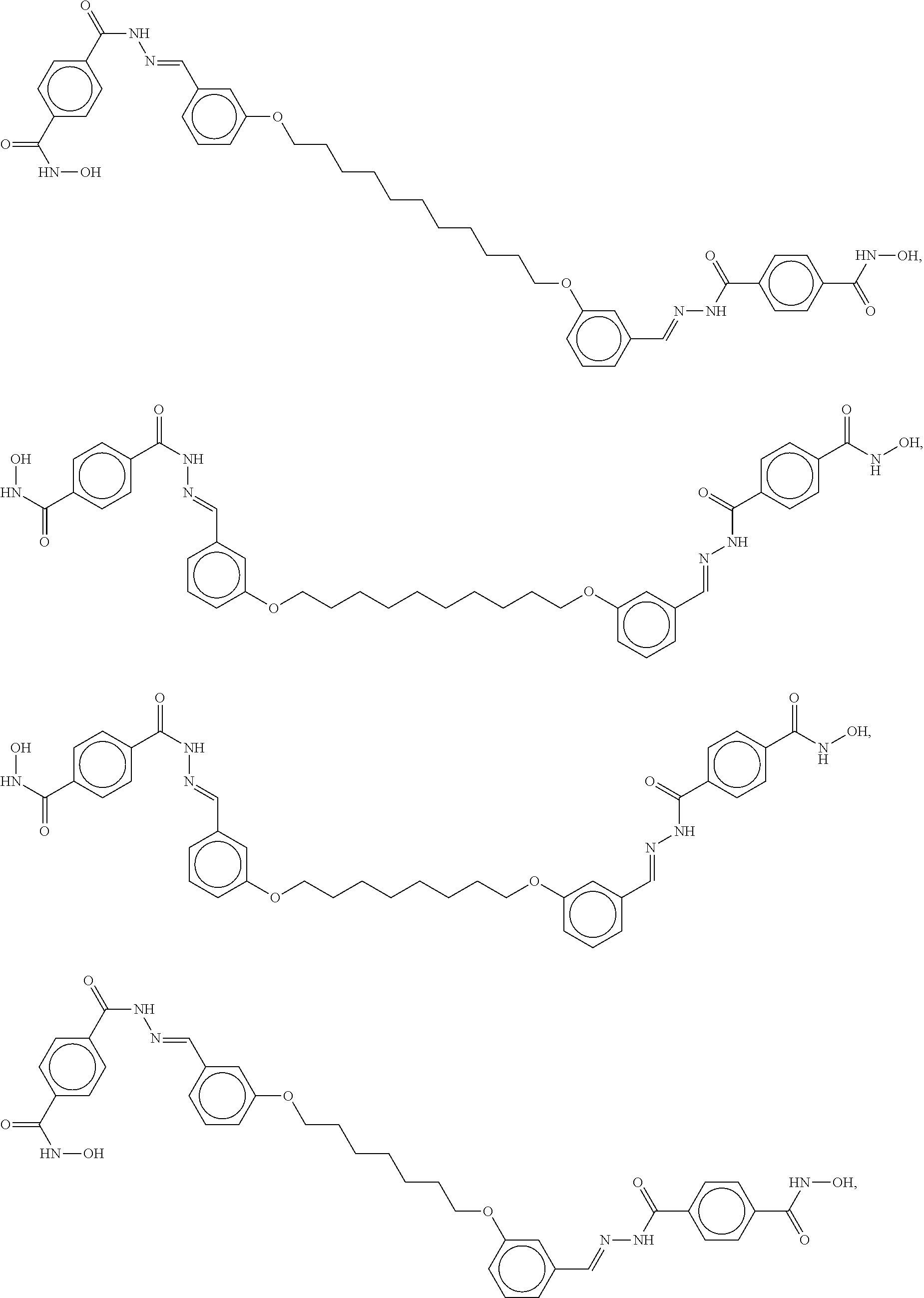

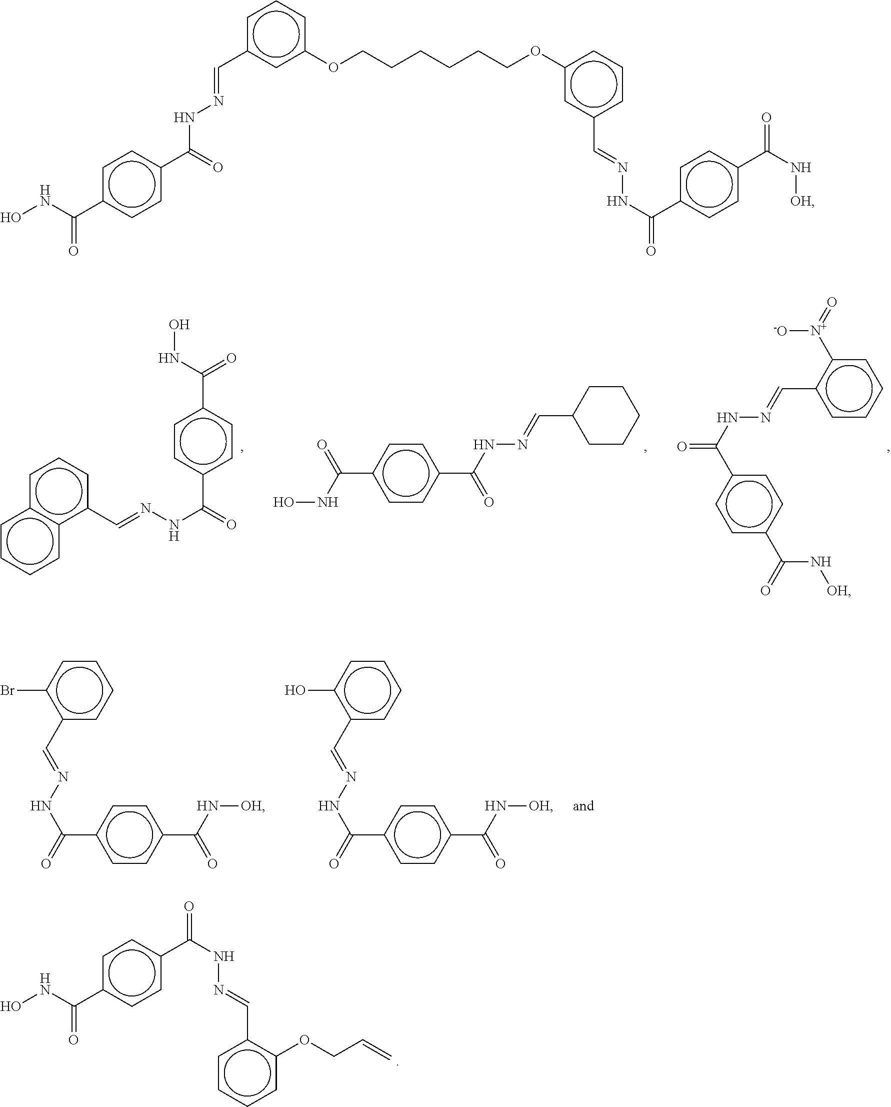

Suitable compounds of Family XX have the following structures:

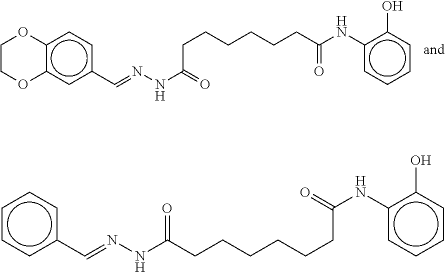

Suitable compounds of Family XXI have the following structures:

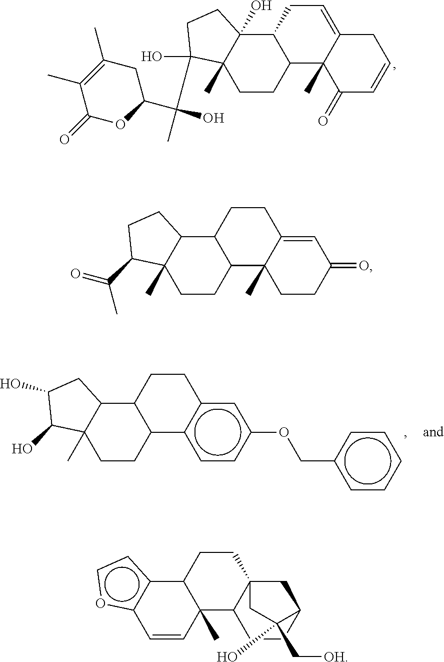

TABLE 2
Other Suitable Compounds

| | |
|---|---|
| 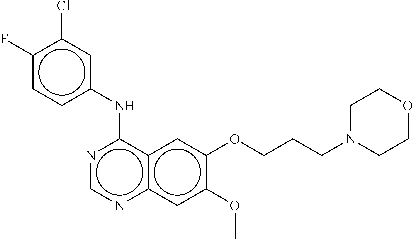 | Compound 1 |
| 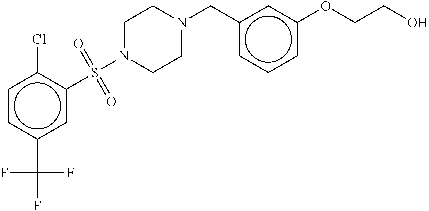 | Compound 2 |
| 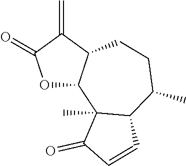 [SMILES: C=C1C(=O)O[C@@H]2[C@H]1CC[C@H](C)[C@@H]1C=CC(=O)[C@@]21C] | Compound 3 |
| 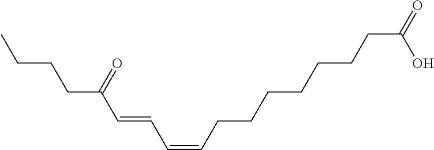 [SMILES: CCCCC(=O)/C=C/C=C\CCCCCCCC(=O)O] | Compound 4 |
| 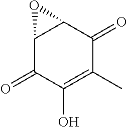 | Compound 5 |
| 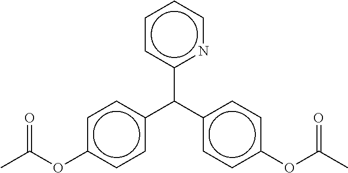 | Compound 6 |
| 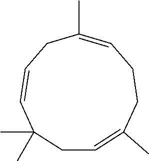 | Compound 7 |
| 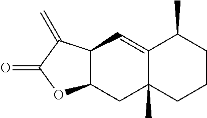 [SMILES: C=C1C(=O)O[C@@H]2C[C@@]3(C)CCC[C@H](C)C3=C[C@H]12] | Compound 8 |
| 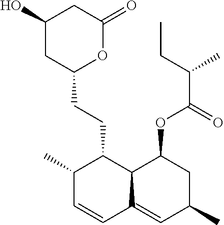 [SMILES: CC[C@H](C)C(=O)O[C@H]1C[C@@H](C)C=C2C=C[C@H](C)[C@H](CC[C@@H]3C[C@@H](O)CC(=O)O3)[C@H]21] | Compound 9 |
| 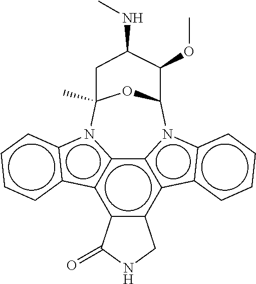 | Compound 10 |
| 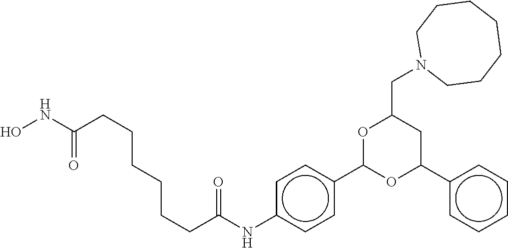 | Compound 11 |
| 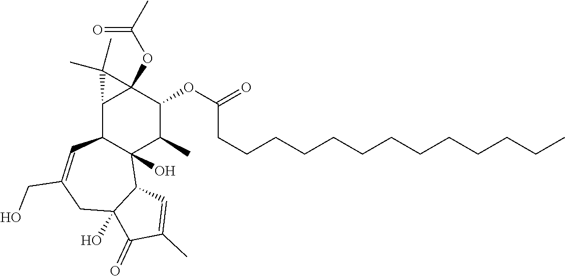 [SMILES: CCCCCCCCCCCCCC(=O)O[C@@H]1[C@@H](C)[C@@]2(O)[C@@H](C=C(CO)C[C@]3(O)C(=O)C(C)=C[C@@H]23)[C@@H]2C(C)(C)[C@]12OC(C)=O] | Compound 12 |
| 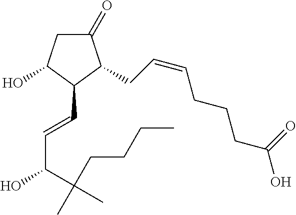 [SMILES: CCCCC(C)(C)[C@H](O)/C=C/[C@H]1[C@H](O)CC(=O)[C@@H]1C/C=C\CCCC(=O)O] | Compound 13 |
| 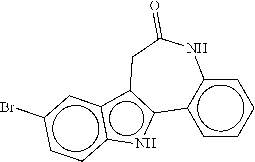 | Compound 14 |
| 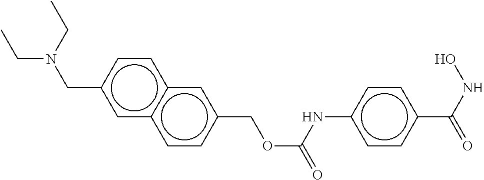 | Compound 15 |
| 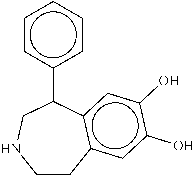 | Compound 16 |
| 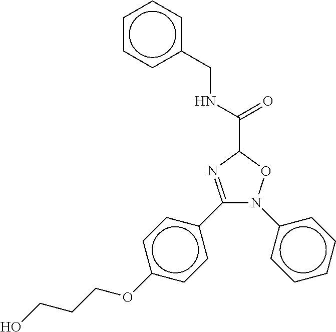 | Compound 17 |

The compounds of the present invention can be administered orally, parenterally, for example, subcutaneously, intravenously, intramuscularly, intraperitoneally, by intranasal instillation, by inhalation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention may also be administered directly to the airways in the form of a dry powder. For use as a dry powder, the compounds of the present invention may be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants and co-solvents and may be filled into conventional aerosol containers that are closed by a suitable metering valve.

### EXAMPLES

### Example 1

### Cell Lines

B16 murine melanoma cells expressing firefly luciferase were used as the parental line for experiments described herein (Murakami et al., Cancer Res. 62:7328 (2002), which is hereby incorporated by reference in its entirety). Human melanoma UACC 1273 and M92047 cell lines are as described in Bittner et al., Nature 406:536 (2000), which is hereby incorporated by reference in its entirety). The human melanoma cell lines Mel375, A2058, Mel 29.6 and Mel501 were obtained from Fred Hutchinson Cancer Research Institute; Seattle, Wash. The murine cell line HT22, a subclone of the HT4 hippocampal cell line, was obtained from The Salk Institute for Biological Studies. Sequences for human WNT3A and WNT5A were amplified by polymerase chain reaction (PCR) and cloned into third generation lentiviral vectors derived from backbone vectors (Dull et al., J. Virol. 72:8463 (1998), which is hereby incorporated by reference in its entirety). These lentiviral vectors contained an EF 1-alpha promoter driving a bi-cistronic message encoding human Wnt isoforms plus GFP. Cells were sorted by fluorescence activated cell sorting (FACS) for GFP expression, with the goal of obtaining cells with approximately equivalent levels of GFP expression.

### Example 2

### Cell Culture

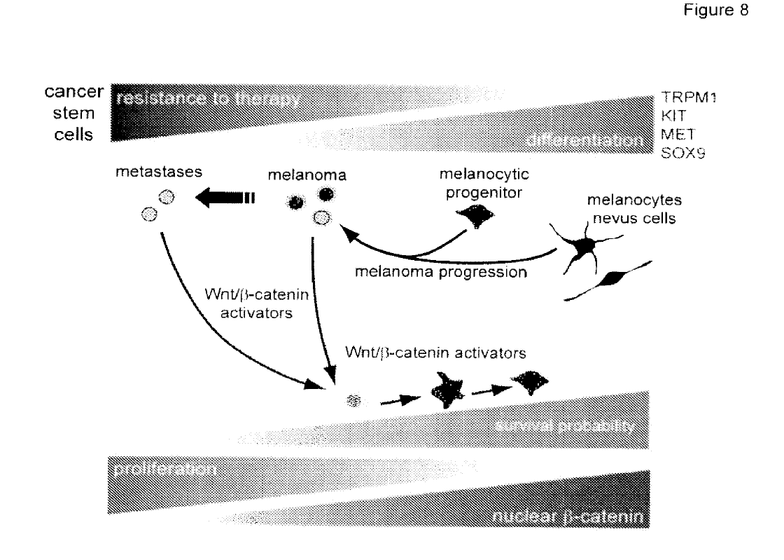

B16 murine melanoma cells were cultured in Dulbeccos modified Eagle's media (DMEM) supplemented with 2% Fetal Bovine Serum, and 1% antibiotic/antimycotic (Invitrogen; Grand Island, N.Y.) (Murakami et al., Cancer Res. 62:7328 (2002), which is hereby incorporated by reference in its entirety). The human melanoma lines Mel375, M92047, A2058, Mel 29.6, Mel501 and Mel526 were cultured in DMEM supplemented with 2% FBS and 1% antibiotic/antimycotic. UACC 1273 cells were cultured in RPMI (Invitrogen; Grand Island, N.Y.) supplemented with 2% FBS and 1% antibiotic/antimycotic. All cell lines were cultured in the presence of 0.02% Plasmocin (InvivoGen; San Diego, Calif.). Synthetic siRNAs (Invitrogen; Grand Island, N.Y.) were transfected into cultured cells at a final concentration of 20 nM using Lipofectamine 2000 (Invitrogen; Grand Island, N.Y.). HT22 cells were cultured in DMEM supplemented with 10% FBS and 1% antibiotic/antimycotic. Sequences for β-catenin siRNA are described in FIG. 8.

### Example 3

### Conditioned Media and Measurement of Wnt Pathway Activation Using a Reporter Assay

Conditioned media was collected from sub-confluent melanoma cell lines, and this media was tested for its ability to activate Wnt/β-catenin signaling in UACC 1273 cells stably transduced with a previously described Wnt/β-catenin-responsive firefly luciferase reporter and a constitutive Renilla luciferase gene used for normalization (Major et al., Science 316:1043 (2007), which is hereby incorporated by reference in its entirety). Conditioned media from B16 melanoma cells was spun down to clear cell debris and then incubated with reporter cells overnight. Activation of the Wnt/β-catenin reporter was measured using a dual luciferase reporter (DLR) assay kit (Promega; Madison, Wis.).

### Example 4

### RNA Purification from B16 Melanoma Cells and PCR Analysis

Cells were cultured for approximately 72 hours until they reached 80-90% confluency. RNA was purified using the RNeasy kit using the manufacturer's protocol (Qiagen; Maryland, Md.). cDNA was synthesized using Superscript Reverse Transcriptase (Invitrogen; Grand Island, N.Y.). Light Cycler FastStart DNA Master SYBR Green1 (Roche; Mannheim, Germany) was used for real-time PCR as previously described (Major et al., Science 316:1043 (2007), which is hereby incorporated by reference in its entirety). Quantitative PCR results presented in the manuscript are representative of experiments performed on a minimum of three biologic replicates.

### Example 5

### Cell Proliferation Assays

For cell counts by hematocytometer, cells were seeded at a uniform density (usually between 10,000 to 25,000 cells per well) in a 12 or 24 well tissue culture plate in the appropriate media. At the end of 3-7 days, cells were trypsinized, resuspended in the appropriate media and counted. Dead cells were identified by 0.4% Trypan Blue stain and excluded from hematocytometer measurements. Cell proliferation experiments were performed with a minimum of six biologic replicates. Similar results were observed for all cell lines using the MTT assay (ATCC; Manassas, Va.), performed according to manufacturer's protocol. For relative cell proliferation assays of B16: GFP cells incubated with lithium chloride or sodium chloride, cell proliferation was measured by luciferase assay. Cell cycle analysis was performed using DAPI-staining and flow cytometry. The Ki-67 rabbit monoclonal antibody was purchased from ThermoFisher (catalog no. RM-9106).

### Example 6

### Immunohistochemistry and Immunoblotting Studies

A polyclonal rabbit anti-β-catenin antibody was used for detection of 1β-catenin (1:1000 dilution for immunoblot, 1:200 dilution for immunohistochemistry). Cells were grown on 18 mm glass coverslips, for 48-72 hours, fixed using 4% paraformaldehyde, permeabilized using 0.25% Triton X-100, and then blocked with 10% goat serum. Goat anti-rabbit Alexa Fluor-568 antibody (Molecular Probes; Eugene, Oreg.) was diluted 1:1000. Cells were counterstained for nucleic acid with DAPI (Molecular Probes; Eugene, Oreg.). Paraffin-embedded nevus sections were stained using an antibody dilution of 1:200. Cellular lysates were obtained by lysing cells on plate with a 0.1% NP-40 based buffer and analyzed by NuPage 4-12% gradient gels (Invitrogen; Grand Island, N.Y.). The WNTSA antibody was obtained from Cell Signaling Technologies (Danvers, Mass.).

### Example 7

### Tumor Microarrays

Tumor microarrays were assembled at the Yale Tissue Microarray Facility. Staining and scoring of tissue microarrays was performed using automated quantification (AQUA) as previously described (Camp et al., Nat. Med. 8:1323 (2002), which is hereby incorporated by reference in its entirety). Statistical analysis, including Kaplan-Meier survival probabilities, ANOVA, and t-tests, was performed using the GraphPad Prism software package (GraphPad Software; La Jolla, Calif.).

### Example 8

### cDNA Microarrays

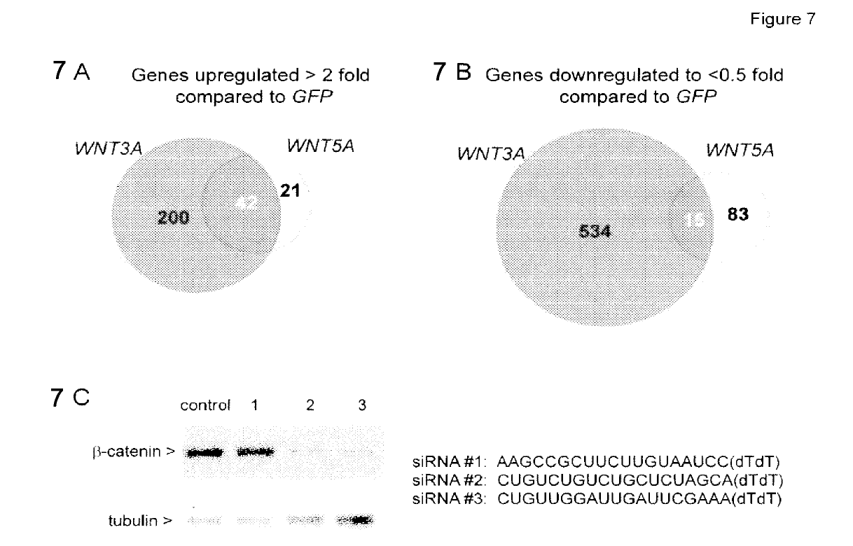

Agilent whole mouse genome array analysis was performed through the microarray core facility at the Huntsman Cancer Institute (Salt Lake City, Utah). Data analysis, including the t-test (Pan, Bioinformatics 18:546 (2002), which is hereby incorporated by reference in its entirety) was performed using the TM4 microarray software suite, which is freely available online (Saeed et al., Biotechniques 34:374 (2003), which is hereby incorporated by reference in its entirety). Two-channel hybridizations were performed with labeled cDNA isolated from three biologic replicates each for cells expressing either WNT3A or WNT5A, using cDNA from GFP-expressing cells as the reference sample. These studies revealed gene sets regulated in both WNT3A and WNT5A cells (FIG. 7), which were then filtered to obtain the top 10% of most variant genes in the WNT3A and WNT5A datasets. Subsequently, an unpaired two-tailed t-test analysis was used to identify genes that were significantly different between the most variant genes in the WNT3A and WNT5A replicate samples, using an arbitrary p-value of p<0.04 as a cut-off. The rationale for further comparing the regulated genes in WNT3A cells to those in WNT5A cells was based the finding that WNT5A did not have significant phenotypic effects (pigmentation, proliferation or cell cycle), and this subsequent comparison allowed identification of potentially important genes regulated by WNT3A that might be missed by setting arbitrary cut-off values for significant genes (i.e. 2-fold upregulated or 50% downregulated).

### Example 9

### High Throughput Small Molecule Screen

Compounds were dissolved in dimethylsulphoxide (DMSO). For the primary screen, performed in duplicate, HT22 cells stably expressing the beta-catenin activated reporter (BAR) were cultured in growth medium (DMEM/10% FBS/1% antibiotic). 3000 cells per well were transferred to 384-well clear bottom plates (Nalgene Nunc; Rochester, N.Y.) in 30 uL of growth medium. The following day, 100 nL of compound and 10 μL of either growth media or WNT3A conditioned media (E.C.₅₀ dose) was transferred to the cells. The next day each well was imaged using transmitted light with the ImageXpress Micro (Molecular devices; Sunnyvale, Calif.) followed by the addition of 10 μL of Steady-Glo (Promega; Madison, Wis.) as per the manufacture's instructions, and luminescence measurement on an EnVision Multilabel plate reader (PerkinElmer; Waltham, Mass.). Viability was scored by analyzing the ImageXpress images. As described in detail in Seiler et al. (Seiler et al., Nucleic Acids Res. 36:D351 (2008), which is hereby incorporated by reference in its entirety), each compound well received an algebraically signed Z-score corresponding to the number of standard deviations it fell above or below the mean of a well-defined mock-treatment distribution of DMSO controls. Z-score normalized data from the growth media stimulus group were sorted by average percent change. The fold-increase over the background of DMSO controls for each treatment was also calculated. The top 50 compounds with the greatest percent change of activity with the growth media were then resorted based on the percent change with the WNT3A stimulus.

### Example 10

### Nuclear β-Catenin Correlates with Improved Patient Survival

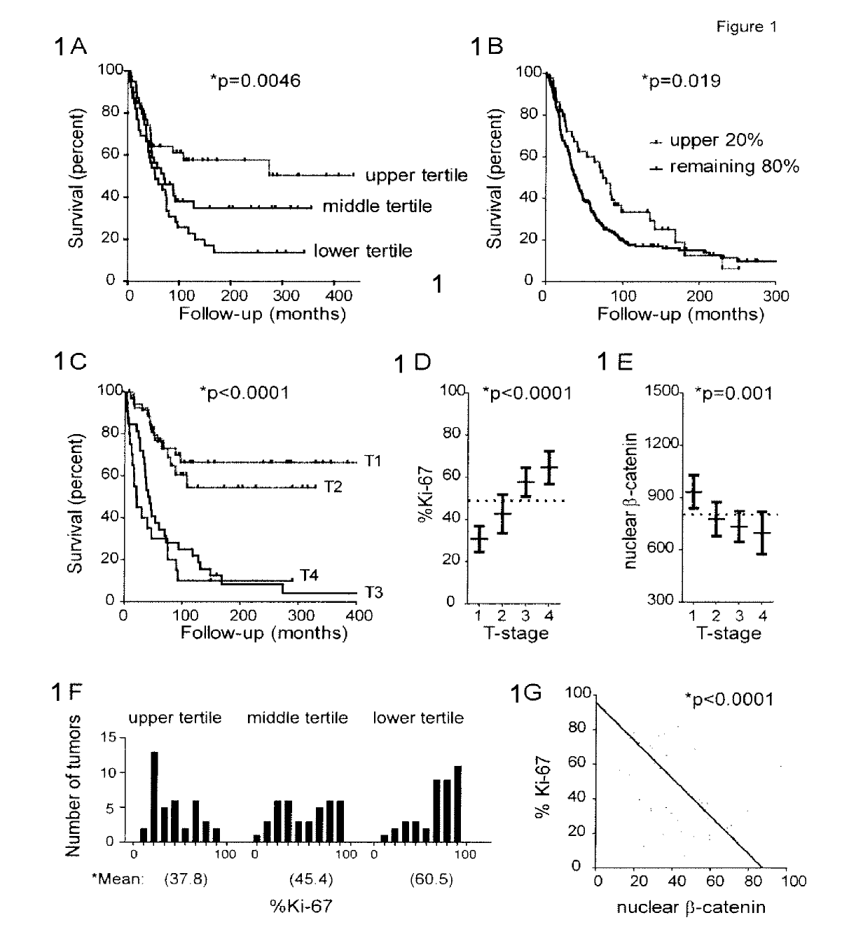

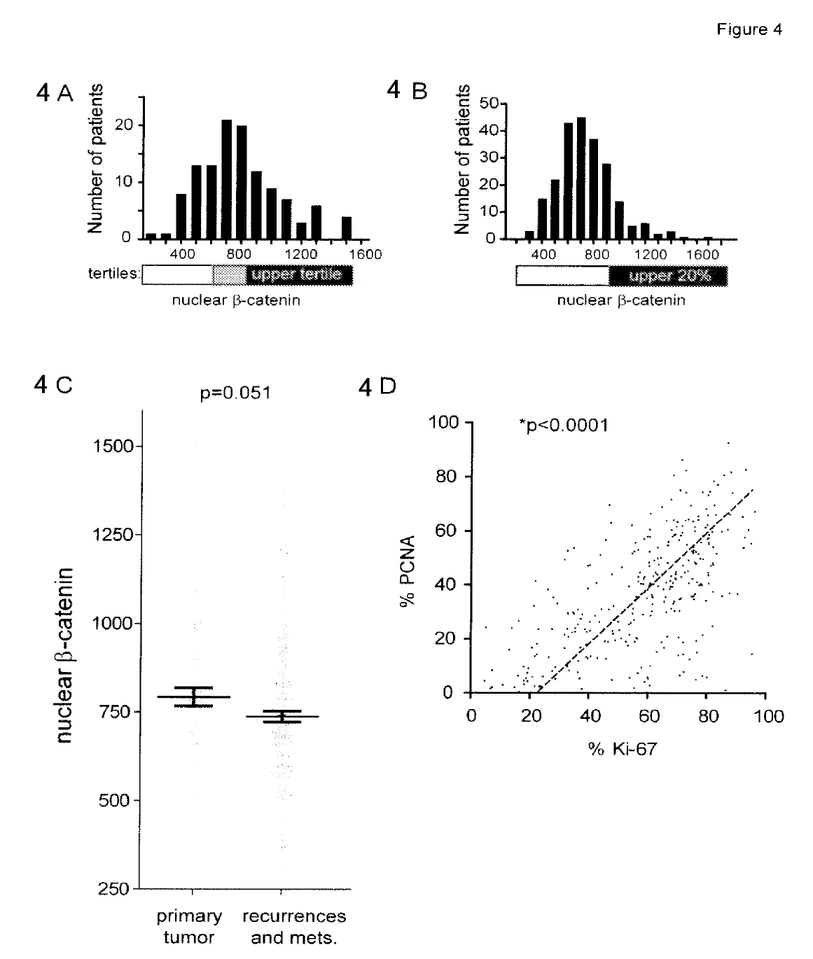

Using the expression of nuclear β-catenin as a clinical surrogate marker for Wnt/β-catenin activation (Bachmann et al., Clin. Cancer Res. 11:8606 (2005); T. Kageshita et al., Br. J. Dermatol. 145:210 (2001); Maelandsmo et al., Clin. Cancer Res. 9:3383 (2003), which are hereby incorporated by reference in their entirety), a tumor microarray composed of 343 cores (118 primary tumors, 225 metastases) from patient tumors (Camp et al., Nat. Med. 8:1323 (2002), which is hereby incorporated by reference in its entirety) was scored. Survival probabilities for patients were estimated using Kaplan-Meier analysis after stratifying primary tumors into tertiles based on nuclear β-catenin expression (FIG. 4). This analysis reveals that higher expression of nuclear β-catenin in both primary tumors (FIG. 1A) and in metastases and recurrences (FIG. 1B) predicts significantly increased patient survival. Also, levels of nuclear β-catenin are lower in metastases and recurrences compared to primary tumors (FIG. 4). These findings confirm and extend previous reports of improved prognosis with elevated nuclear β-catenin in melanoma (Bachmann et al., Clin. Cancer Res. 11:8606 (2005); T. Kageshita et al., Br. J. Dermatol. 145:210 (2001); Maelandsmo et al., Clin. Cancer Res. 9:3383 (2003), which are hereby incorporated by reference in their entirety).

### Example 11

### Nuclear β-Catenin is Negatively Correlated with Proliferation

As tumor depth measurements (Breslow thickness) were obtained for 113 primary tumors in our array cohort, this sub-group of patients was analyzed based on the Breslow thickness stratification used as reported (Thompson, J. A., Semin. Oncol. 29:361 (2002), which is hereby incorporated by reference in its entirety). Increasing tumor depth is correlated with a lower probability of survival (FIG. 1C) and with a higher degree of proliferation, which is measured by the percentage of cells expressing Ki-67 (FIG. 1D). By contrast, nuclear β-catenin levels are highest for shallow tumors (T1) and decrease significantly with increased tumor depth (FIG. 1E).

The percentage of tumors staining positive is then analyzed for the cellular proliferative marker Ki-67 (% Ki-67). Strikingly, distribution histograms of % Ki-67 staining in primary tumors stratified by expression of nuclear β-catenin show a statistically significant shift towards increased proliferation (elevated % Ki-67 staining) in the groups with lower nuclear β-catenin (FIG. 1F). It is shown that there is no correlation between expression of β-catenin and % Ki-67 staining, and PCNA is used as an independent marker of proliferation (FIG. 4). Taken together these data demonstrate that elevated nuclear β-catenin is negatively associated with proliferation as measured by either tumor size/depth, or by the markers Ki-67 and PCNA.

### Example 12

### Activation of Wnt/β-Catenin Signaling Changes Melanoma Cell Fate

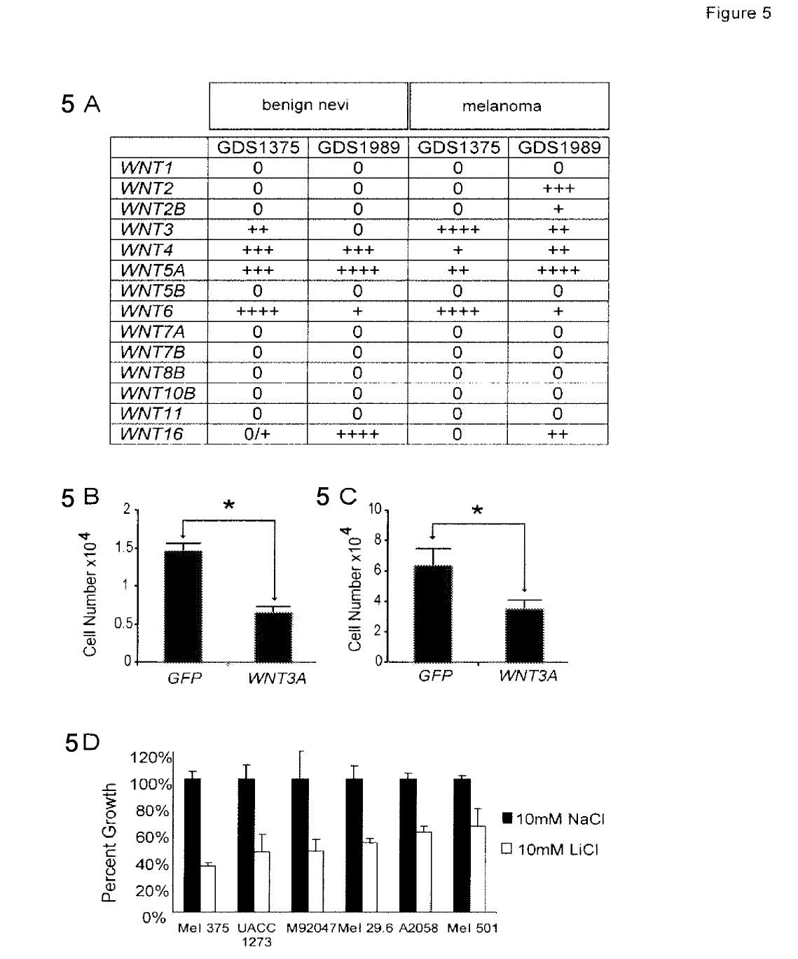

Wnts, which can activate or antagonize β-catenin signaling, were investigated in order to elicit changes in melanoma cells cultured in vitro that might be consistent with the above clinical data. Since melanoma tumors appear to express WNT3A (FIG. 5), which has a pivotal role in the regulation of melanocyte biology (Dorsky et al., Genes Dev. 14:158 (2000); Fang et al., Stem Cells 24:1668 (2006), which are hereby incorporated by reference in their entirety), and they express WNTSA, which is elevated in melanoma metastases (Bittner et al., Nature 406:536 (2000); Weeraratna et al., Oncogene 23:2264 (2004), which are hereby incorporated by reference in their entirety), B16 mouse melanoma cells were transduced with lentivirus constructs encoding WNT3A, WNT5A, or a GFP control.

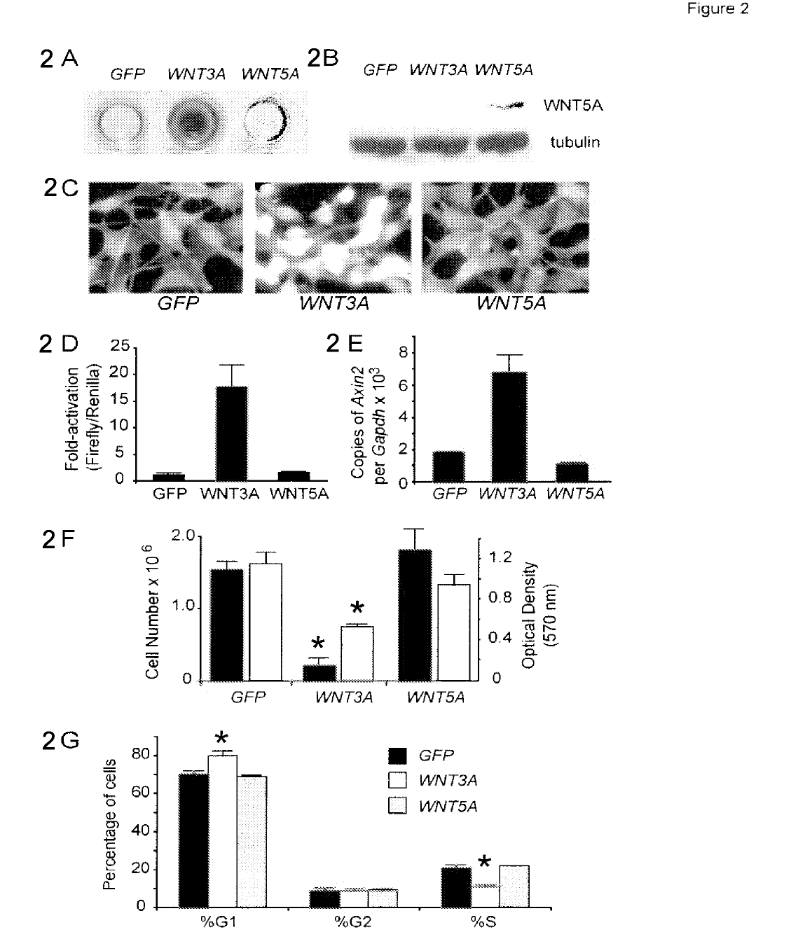

B16:WNT3A cells exhibit strikingly increased pigmentation compared to GFP or WNT5A cells (FIG. 2A). Scoring cells for nuclear accumulation of β-catenin revealed that only cells expressing WNT3A, and not WNT5A or GFP, exhibit elevated β-catenin (FIG. 2C). As a positive control it was shown that conditioned media (CM) from BI 6 cells expressing WNT3A activates a β-catenin-responsive reporter in UACC 1273 melanoma cells (FIG. 2D), confirming that these cells were secreting active WNT3A. Also, it was shown that B16 cells expressing WNT3A exhibit marked increases in expression of the β-catenin target gene Axin2 (Jho et al., Mol. Cell Biol. 22:1172 (2002), which is hereby incorporated by reference in its entirety) compared to B16:GFP cells (FIG. 2E).

In vitro cell proliferation studies using the MTT cell proliferation assay showed that B16 cells expressing WNT3A exhibit decreased proliferation compared to cells expressing GFP or WNT5A (FIG. 2F). This finding was paralleled in human cell lines (FIG. 5). Cell cycle profiles were then compared to the Wnt-transduced melanoma cell lines, and found that cells expressing WNT3A exhibit an increased population in G1, with a decreased population in S phase, compared to control cells (FIG. 2G). Together, these data suggest that WNT3A can induce differentiation of the melanoma cells to a cell fate that is more pigmented and less proliferative.

### Example 13

### Elevation of Melanocyte Differentiation Markers by WNT3A

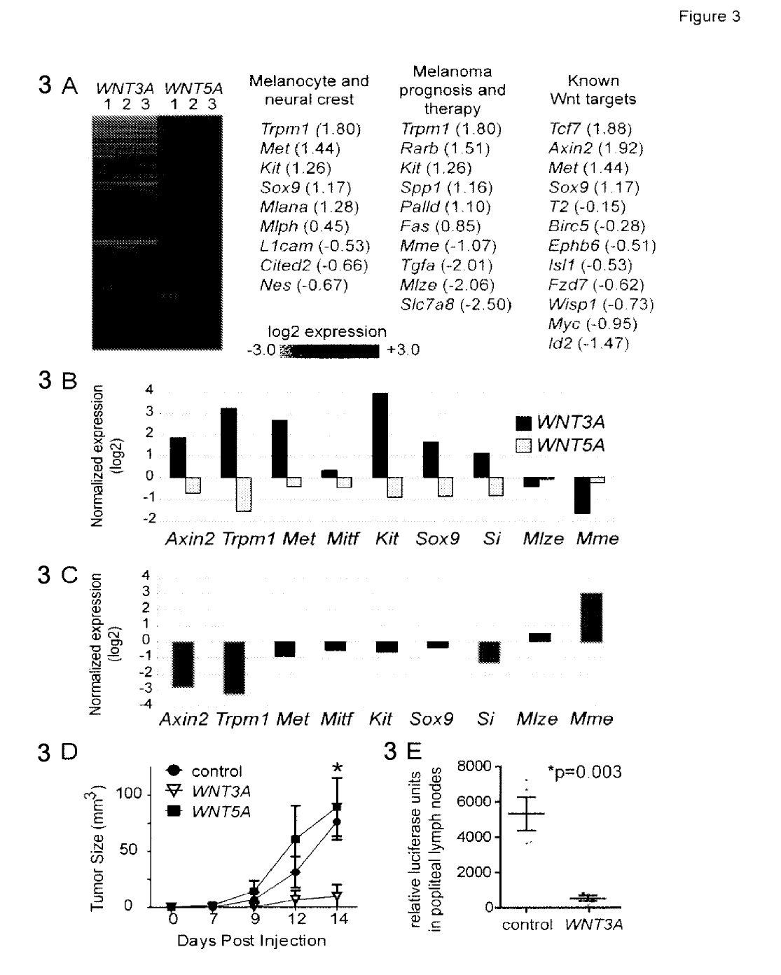

Next, a genome-wide transcriptional profiling was performed to gain further insights into the consequences of expression of WNT3A and WNT5A, which revealed that levels of transcripts elevated by WNT3A were actually reduced by WNT5A (FIG. 3B). Among the most highly significant genes elevated by WNT3A (FIG. 3A) are Axin2 (Jho et al., Mol. Cell Biol. 22:1172 (2002), which is hereby incorporated by reference in its entirety) and Tc7 (Roose et al., Science 285:1923 (1999), which is hereby incorporated by reference in its entirety), which are direct targets of Wnt/β-catenin signaling; Mme and Mize, downregulated genes previously linked to melanoma progression (Watabe et al., Jpn. J. Cancer Res. 92:140 (2001); Bilalovic et al., Mod. Pathol. 17:1251 (2004), which are hereby incorporated by reference in their entirety); Mitf, linked to pigment cell fate, and Trpm1, Met, Sox9 and Kit, which are highly expressed during melanocyte and neural crest development (Loftus et al., Proc. Natl. Acad Sci. USA 96:9277 (1999, which is hereby incorporated by reference in its entirety)). To confirm the array data levels of selected transcripts were measured by quantitative PCR (FIG. 3B). To establish that the effects of WNT3A on gene expression were specific, it was demonstrated that the changes in gene expression were antagonized by β-catenin siRNA (FIG. 3C). The transcriptional profiling thus supports the conclusion, evident from visual examination of cells (FIG. 2A), that WNT3A promotes melanoma cells adopting characteristics of melanocyte differentiation.

### Example 14

### WNT3A Reduces Melanoma Tumor Size and Metastasis in Mice

While expression of Trpm1 was elevated by WNT3A (FIG. 3B), its expression is usually reduced during melanoma progression. Taken with the observed changes in cell fate and proliferation seen in cells expressing WNT3A, this led to the prediction that cells expressing WNT3A would form less proliferative and less aggressive tumors in vivo. Indeed, implantation of WNT3A-transduced B16 cells into the footpads of C57BL/6 mice, significantly decreased tumor growth compared to B16 cells transduced with GFP or WNT5A (FIG. 3D) and decreased metastases to popliteal lymph nodes (FIG. 3E).

### Example 15

### A High-Throughput Screen for Therapeutic Activators of Wnt/β-Catenin Signaling

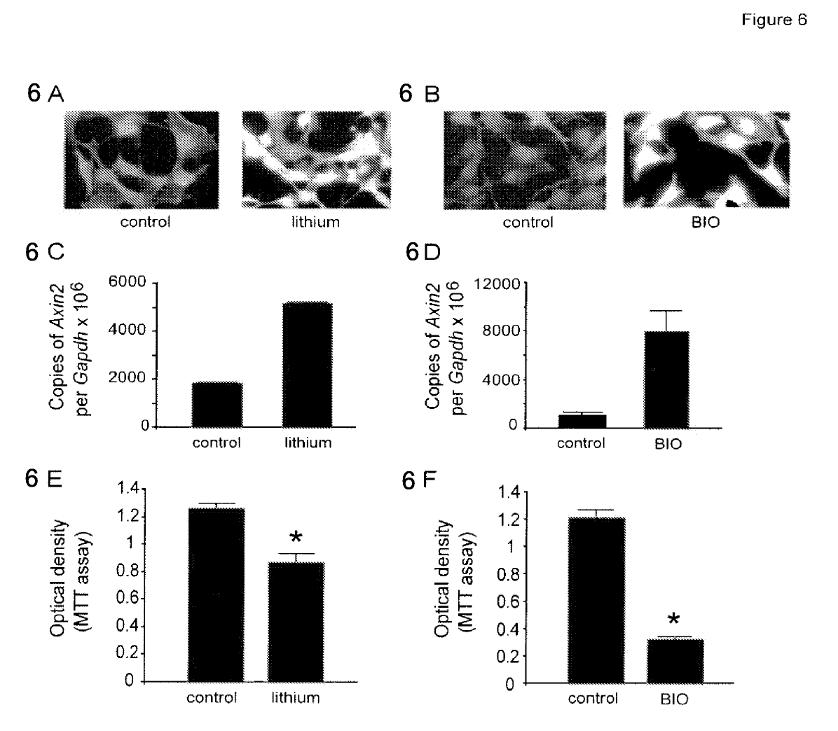

In support of the hypothesis that activation of Wnt/β-catenin inhibits melanoma growth, treatment of B16 cells with the GSK3 inhibitors lithium chloride (LiCl) or 6-bromoindirubin-3′-oxime (BIO) also resulted in decreased proliferation of cultured cells (FIG. 6). Precluding further consideration of either compound in murine therapeutic trials it was found that lithium and BIO exhibit several obstacles relating to both toxicity and, in the case of lithium, to difficulty maintaining adequate serum levels in mice. Consequently, identifying novel activators of Wnt/β-catenin signaling that would have improved therapeutic efficacy and tolerance became a target.

A high-throughput screen of >60% of the FDA-approved panel of biologically active small molecules was performed using a Wnt-responsive luciferase reporter system to identify compounds that could either activate Wnt/β-catenin signaling on their own, or synergize with WNT3A to enhance reporter activation.

Data in support of a novel “differentiation therapy” for treating melanoma with agents that activate β-catenin signaling are presented in FIG. 8. Mechanistically, it is proposed that activating Wnt/β-catenin signaling forces melanoma cells to adopt a more differentiated cell fate, resembling melanocytes, which are intrinsically less motile, less proliferative, and thus less deadly. The concept that different states of differentiation and pluripotency exist in melanoma is not new, and in fact recent research has focused on markers that may better identify so-called melanoma stem cells, or tumor initiating cells (Zabierowski et al., Cancer Cell 13:185 (2008); Schatton et al., Nature 451:345 (2008); Grichnik et al., J. Invest. Dermatol. 126:142 (2006), which are hereby incorporated by reference in their entirety). The forced differentiation of these tumor initiating populations into cell fates that are either more benign (i.e. slower growing, or less metastatic) or more treatable provides a new approach that could prove beneficial in concert with current therapies based on cytotoxicity or immunomodulation.

For the activation of β-catenin signaling to be considered as a therapy one would need reasonable assurance that enhancing Wnt/β-catenin signaling in melanoma will not have the undesirable consequence of promoting proliferation. In support of the present data, a recent study found that expression of a stabilized β-catenin mutant (β-cat^{STA}) in mice did not increase proliferation of melanocytic cells, which is entirely consistent with our findings (Delmas et al., Genes Dev. 21:2923 (2007), which is hereby incorporated by reference in its entirety). This study also found that restricting the expression of β-cat^{STA} to melanocytes did not lead to any melanomas over a 2-year period (Delmas et al., Genes Dev. 21:2923 (2007), which is hereby incorporated by reference in its entirety). Additionally, other published reports (Ballin et al., Br. J. Cancer 48:83 (1983); Penso et al., Mol. Genet. Metab. 78:74 (2003); Kang et al., Arch. Dermatol. Res. 294:426 (2002), which are hereby incorporated by reference in their entirety) confirm our observed inhibition of melanoma cell proliferation upon treatment with lithium chloride, which is a pharmacologic activator of Wnt/β-catenin signaling. Available data therefore strongly suggest that activation of Wnt/β-catenin signaling is not by itself oncogenic in the context of melanoma.

Herein, it is suggested that future studies should consider a combination therapy in which activation of Wnt/β-catenin signaling forces higher expression of Kit, which may increase their sensitivity to imatinib or sunitinib. Given that current therapeutic strategies have proven largely ineffective for metastatic melanoma, a “differentiation therapy” involving activators of β-catenin signaling, used as monotherapy or in combination therapy, may provide a new alternative for treating this disease.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a subject for a condition mediated by aberrant Wnt/β-catenin signaling, said method comprising:
selecting a subject with a condition mediated by aberrant Wnt/β-catenin signaling and
administering to the selected subject a compound selected from the group consisting of those set forth in Table 1, Table 2, and a pharmaceutically acceptable salt thereof.

2. The method of claim 1, wherein the subject is human.

3. The method of claim 1, wherein a compound of Table 1 or a pharmaceutically acceptable salt thereof is administered.

4. The method of claim 1, wherein a compound of Table 2 or a pharmaceutically acceptable salt thereof is administered.

5. The method of claim 1, wherein the condition is selected from the group consisting of cancer, bone mass diseases, fracture repair, FEVR, diabetes mellitus, cord blood transplants, psychiatric disease, neurodegenerative disease, hair loss, diseases linked to loss of stem/progenitor cells, conditions improved by increasing stem/progenitor cell populations, HIV, and tooth agenesis.

6. A method of activating the Wnt/β-catenin pathway in a subject comprising:
selecting a subject in need of Wnt/β-catenin pathway activating and
administering to the selected subject a compound selected from the group consisting of those set forth in Table 1, Table 2, and a pharmaceutically acceptable salt thereof.

7. The method of claim 6, wherein a compound of Table 1 or a pharmaceutically acceptable salt thereof is administered.

8. The method of claim 6, wherein a compound of Table 2 or a pharmaceutically acceptable salt thereof is administered.


SEQUENCE LISTING (3 sequences)

SEQ ID NO: 1 - DNA, 21 bp, Artificial Sequence
  Description of Artificial Sequence Synthetic oligonucleotide
aagccgcuucuuguaaucctt

SEQ ID NO: 2 - DNA, 21 bp, Artificial Sequence
  Description of Artificial Sequence Synthetic oligonucleotide
cugucugucugcucuagcatt

SEQ ID NO: 3 - DNA, 21 bp, Artificial Sequence
  Description of Artificial Sequence Synthetic oligonucleotide
cuguuggauugauucgaaatt